(19) United States
(12) Patent Application Publication
(10) Pub. No.: US 2004/0171109 A1
(43) Pub. Date: Sep. 2, 2004

(54) IL-17 receptor-like protein, uses thereof, and modulation of catabolic activity of il-17 cytokines on bone and cartilage

(76) Inventors: Dominik Haudenschild, Davis, CA (US); Larry Rose, Carmichael, CA (US); Timothy Moseley, Davis, CA (US); A. Hari Reddi, El Macero, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/416,442

(22) PCT Filed: Nov. 13, 2001
(86) PCT No.: PCT/US01/43855

Related U.S. Application Data
(60) Provisional application No. 60/247,134, filed on Nov. 10, 2000.
(60) Provisional application No. 60/271,197, filed on Feb. 23, 2001.
(60) Provisional application No. 60/328,904, filed on Oct. 12, 2001.

(51) Int. Cl. C07K 14/715; C07H 21/04

(52) U.S. Cl. 435/69.1; 435/320.1; 435/325; 530/350; 536/23.5

(57) ABSTRACT

This invention relates, first, to the discovery of IL-17RL, a receptor for members of the IL-17 cytokine family, particularly IL-17B. IL-17RL is naturally expressed from a 19-exon gene as a 720 amino acid protein and as any of 11 splice variants created from differential splicing of the 19 exons. The invention further relates to the discovery that IL-17B is involved in catabolic degradation of bone and cartilage and that bone and cartilage disorders can be ameliorated by pharmacological manipulation of IL-17B by antibodies or by using IL-17RL as a decoy to reduce IL-17B related catabolic activity. Methods for diagnosing cartilage disorders, and pharmaceutical compositions for slowing or ameliorating bone and cartilage pathologies are also provided.

### CROSS-REFERENCES TO RELATED APPLICATIONS

This application claims priority from U.S. Provisional Patent Application Nos. 60/247,134, filed Nov. 10, 2000 (attorney docket no. 02307O-115500US), 60/271,197, filed Feb. 23, 2001 (attorney docket no. 02307O-116300US), and 60/328,904, filed Oct. 12, 2001 (attorney docket no.02307O-115510US). The contents of all of these applications are hereby incorporated by reference for all purposes.

### STATEMENT AS TO RIGHTS TO INVENTIONS MADE UNDER FEDERALLY SPONSORED RESEARCH AND DEVELOPMENT

Not Applicable.

### REFERENCE TO A “SEQUENCE LISTING,” A TABLE, OR A COMPUTER PROGRAM LISTING APPENDIX SUBMITTED ON A COMPACT DISK

Not applicable.

### FIELD OF THE INVENTION

This invention relates to the discovery of a new receptor-like protein (“IL-17RL”) for members of the IL-17 family of cytokines, particularly IL-17B (also known as “chondroleukin”). The invention further concerns the discovery that IL-17 cytokines, and particularly IL-17B, is involved in catabolic degradation of bone and cartilage. Antagonists to IL-17 cytokines, such as IL-17RL, can be used to reduce damage to bone and cartilage caused by IL-17 cytokines. Further, the invention relates to the discovery that IL-17RL can be as a diagnostic marker in determining the aggressiveness of prostate cancers.

### BACKGROUND OF THE INVENTION

Interleukins were historically defined as soluble secreted factors expressed in immune cells which mediate interactions between leukocytes. However this definition has evolved to include cytokines with a spectrum of pleiotropic actions (reviewed in Paul et al., Cell, 76(2):241-51 (1994); Arai et al., Annu Rev Biochem, 59:783-836 (1990)). Interleukin-17 is a recently discovered cytokine which exerts its effect on many different tissues due to the nearly ubiquitous distribution of its receptor (Yao et al., J Immunol, 155(12):5483-6 (1995); Spriggs, J Clin Immunol, 17(5):366-9. (1997); Fossiez et al., Int Rev Immunol, 16(5-6):541-51 (1998); Yao et al., Cytokine, 9(11):794-800 (1997); Rouvier et al., J Immunol, 150(12), 5445-56 (1993); Van bezooijen et al., J Bone Miner Res, 14(9), 1513-21 (1999)). IL-17 is a pro-inflammatory cytokine that has been implicated in a number of diseases including rheumatoid arthritis (Chabaud et al. Arthritis Res, 3(3):168-77 (2001); Lubberts et al., Arthritis Rheum, 43(6):1300-6 (2000); Miossec, Curr Opin Rheumatol, 12(3):181-5 (2000)), allergic skin immune response (Albanesi et al., J Immunol, 165(3):1395-402 (2000)), organ transplant rejection (Antonysamy et al., Transplant Proc, 31(1-2):93 (1999); Antonysamy et al., J Immunol, 162(1):577-84 (1999); Loong et al., Transplant Proc, 32(7):1773 (2000); Van Kooten et al., J. Am Soc Nephrol, 9(8):1526-34 (1998)), and multiple sclerosis (Matusevicius et al., Mult Scler, 5(2):101-4 (1999)). Recent work has identified four related proteins, establishing an IL-17 family of cytokines. These new family members, IL-17B (also known as “chondroleukin”), IL-17C, IL-17E, and IL-17F, share 20-30% homology with IL-17, and have 4 conserved cysteines and all contain a putative N-terminal signal peptide typically required for secretion (Li et al., Proc Natl Acad Sci USA, 97(2):773-8 (2000); Lee et al., J Biol Chem, 276(2):1660-4 (2001); Shi et al., J Biol Chem, 275(25):19167-76 (2000)). Reflecting the discovery of other members of the IL-17 family, the cytokine originally designated as IL-17 has now been redesignated by some researchers as “IL-17A.”

The IL-17 signaling pathway is currently being studied. Although it is not a kinase itself, the IL-17 receptor (“IL-17R”) has been shown to transduce its signal through the activation of ERK, JNK/SAPK, and p38 MAP kinase pathways (Shalom-Barak et al., J Biol Chem, 273(42):27467-73 (1998); Subramaniam et al., Biochem Biophys Res Commun, 262(1):14-9 (1999); Martel-Pelletier et al., Arthritis Rheum, 42(11):2399-409 (1999)). In the presence of IL-17 ligand, these pathways lead to the up-regulation of genes typically associated with inflammation such as stromelysin, IL-6, and IL-1β, and the activation of NF-kB (Awane et al., J. Immunol, 162(9):5337-44 (1999); Kehlen et al., J Neuroimmunol, 101(1):1-6 (1999); Broxmeyer, J Exp Med, 183(6):2411-5 (1996)).

Recently, a receptor was identified as a receptor for IL-17B, and was named IL-17BR (Shi et al., J Biol Chem, 275(25):19167-76 (2000)). This receptor was subsequently shown, however, to have much greater affinity for IL-17E than for IL-17B. Accordingly, this receptor is now considered to be an IL-17E receptor, and has been redesignated as IL-17Rhl. This receptor was shown to activate NF-kB in an in vitro luciferase assay (Lee et al., J Biol Chem, 276(2):1660-4 (2001)).

Among the tissues of the musculoskeletal system there is a spectrum of potential for regeneration and repair. Bone has the highest potential for repair; cartilage the least. Bone and cartilage share a common ontogeny, and the growth factors that promote chondrogenesis and osteogenesis overlap. See Reddi, Pediatr Nephrol 14:598-601 (2000). Despite their common lineage, mature cartilage has much less capacity than bone for repair and regeneration. The cellular and molecular basis of this profound difference remain unknown. However, extensive research on bone morphogenetic proteins (BMPs) and bone induction suggests that articular cartilage may contain uncharacterized endogenous inhibitors of repair that limit its regenerative capacity. See Reddi, Nature Biotechnology 16(3):247-52 (1998). Likewise, therapeutic use of BMPs for bone induction has been hampered by the presence of unknown endogenous BMP inhibitors (see Groeneveld & Burger, Eur J Endocrinology 142:9-21 (2000)).

### SUMMARY OF THE INVENTION

The invention provides nucleic acids encoding a polypeptide with 85% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F. In preferred forms, the nucleic acid encodes a polypeptide with 90% or greater sequence identity to the identified sequences and, in still more preferred embodiments, provides nucleic acids encoding polypeptides with at least 95% to such polypeptides. In the most preferred embodiments, the nucleic acid encodes a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

The invention further provides expression cassettes comprising a promoter operatively linked to one of the above-described nucleic acids, as well as host cells comprising such expression cassettes. The host cell can be selected from the group consisting of a chondrocyte, a synoviocyte, and a mesenchymal stem cell.

In a further group of embodiments, the invention provides polypeptides with 85% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F. In more preferred embodiments, the polypeptide has 90% or greater sequence identity to polypeptides having the sequences noted above and, in even more preferred embodiments, has 95% or greater sequence identity to such polypeptides. In the most preferred forms, the polypeptides have a a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

The invention further provides compositions comprising one of the above-described polypeptides and a pharmaceutically acceptable carrier.

In another group of embodiments, the invention provides the use of any of the nucleic acid sequences described above for the manufacture of a medicament to modulate cartilage or bone growth in a mammal. In preferred embodiments, the mammal is a human. The invention further provides the use of any of the polypeptides described above for the manufacture of a medicament to modulate cartilage or bone growth in a mammal. In preferred forms, the mammal is a human.

The invention further provides for the use of any of the nucleic acid sequences described above for the manufacture of a medicament to restore androgen-responsiveness to a prostate cancer cell.

In another group of embodiments, the invention provides a method of decreasing catabolic activity in bone or cartilage in a mammal, said method comprising administering to said mammal a polypeptide with 85% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ B NO:21, SEQ ID NO:23, and SEQ ID NO:25, which polypeptide binds to an interleukin-17 (IL-17) selected from the group consisting of IL-17A, IL-17B, IL-17C, IL-17E and IL-17F, in an amount sufficient to lower levels of said IL-17, thereby decreasing catabolic activity in said bone or cartilage. In more preferred forms, the polypeptide has 90% or greater sequence identity to the sequences named and, in still more preferred forms, has 95% or greater identity to one of these sequences. In the most preferred embodiments, the polypeptide has a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

The invention further provides methods for determining the aggressiveness of a prostate cancer cell. The method comprises determining the presence or absence in the cell of an epitope of IL-17RL (SEQ ID NO:1) wherein a determination that the epitope is absent in the cell indicates that the cell is more aggressive than a like cell in which said epitope is present. The detection of the presence or absence of the epitope is preferably performed using an antibody which specifically binds said epitope.

In yet another set of embodiments, the invention provides methods of restoring androgen-responsiveness to a prostate cancer cell, comprising administering to the prostate cancer cell a nucleic acid encoding a polypeptide which binds IL-17B, and further wherein the polypeptide has 90% or greater sequence identity to a polypeptide having a sequence selected from the group consisting of: SEQ ID NO:2, SEQ ID NO:17, SEQ ID NO:19, SEQ ID NO:21, SEQ ID NO:23 and SEQ ID NO:25. In more preferred forms, the polypeptide has 95% or greater identity to one of these polypeptides and, in the most preferred form, has a sequence selected from that of one of these polypeptides.

The invention further provides antibodies which bind to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25. In some embodiments, it is preferred if the antibody binds to an extracellular domain of the polypeptide.

In still another group of embodiments, the invention provides methods of stimulating growth of bone or cartilage in a patient with a bone or cartilage pathology, the method comprising administering an IL-17B antagonist to the patient, thereby stimulating the growth of bone or cartilage. The method can further comprise the step of administering a tissue graft to the patient. In some embodiments, the invention comprises the step of administering a bone or cartilage growth factor to the patient. The bone or cartilage growth factor can be a bone morphogenetic protein. These methods are especially useful where the patient has a degenerative cartilage disorder. The invention also provides for methods further comprising the steps of obtaining a sample of tissue from the patient, and measuring the amount of IL-17B in the sample. The IL-17B antagonist can be, for example, a polyclonal antibody, a monoclonal antibody, a soluble IL-17B receptor, or an IL-17B receptor immobilized on a surface, such as a gel matrix. The IL-17B antagonist can be IL-17RL or a portion thereof that binds IL-17B. The soluble IL-17B receptor can be an IL-17RL lacking a transmembrane domain. In particular, the IL-17RL lacking a transmembrane domain can be selected from the group consisting of SEQ ID NO:5, SEQ ID NO:7, SEQ ID NO:9, SEQ ID NO:11, SEQ ID NO:13, and SEQ ID NO:15. The IL-17B antagonist can be a polynucleotide encoding a soluble IL-17B receptor. The soluble IL-17B receptor can be, for example, selected from the group consisting of SEQ ID NO:5, SEQ ID NO:7, SEQ ID NO:9, SEQ ID NO:11, SEQ ID NO:13, and SEQ ID NO:15. The IL-17B antagonist can also be a IL-17B antisense polynucleotide.

The invention further provides a method of potentiating the activity of a bone morphogenetic protein in a mammal, the method comprising administering the bone morphogenetic protein and a IL-17B antagonist to the mammal. Further, the invention provides a method of enhancing the regenerative potential of a tissue graft, comprising transforming cells of the tissue graft with an IL-17B antagonist. And, the invention provides a method of inhibiting ossification or calcification in a mammal suffering from pathological ossification or calcification, the method comprising administering IL-17B to the mammal in an amount sufficient to inhibit ossification or calcification. Moreover, the invention provides methods of diagnosing a cartilage degenerative disorder in a mammal, comprising the steps of: obtaining a sample of tissue from the mammal. measuring the amount of IL-17B in the sample, and comparing the amount of IL-17B in the sample with the amount of IL-17B in a sample of tissue from a mammal known to have a cartilage degenerative disorder.

The invention further provides methods of stimulating proteoglycan synthesis by a chondrocyte in culture by contacting the chondrocyte with an IL-17B antagonist, such that the rate of proteoglycan synthesis is increased. The IL-17B antagonist can be a soluble IL-17RL. The soluble IL-17RL can be selected from the group consisting of SEQ ID NO:5, SEQ ID NO:7, SEQ ID NO:9, SEQ ID NO:11, SEQ ID NO:13, and SEQ ID NO:15 The invention further provides a method of inhibiting the rate of proteoglycan synthesis by a chondrocyte in culture, comprising the step of contacting the chondrocyte with IL-17B, such that the rate of proteoglycan synthesis is decreased. The invention further provides for mammalian cells comprising a polynucleotide encoding an IL-17B antagonist, wherein the mammalian cell is selected from the group consisting of a chondrocyte, a synoviocyte, and a mesenchymal stem cell.

The invention further provides compositions comprising a cartilage growth factor and an IL-17B antagonist in a pharmaceutically acceptable carrier, as well as compositions comprising an IL-17B antagonist and an insoluble carrier matrix.

In yet another set of embodiments, the invention provides a use of an IL-17B antagonist selected from the group consisting of an antibody and IL-17RL for the manufacture of a medicament to stimulating growth of bone or cartilage in a patient with a bone or cartilage pathology. The IL-17B antagonist can be a soluble IL-17RL. In some uses, the soluble IL-17RL is a polypeptide with at least 85% sequence identity to a sequence selected from the group consisting of SEQ ID NO:5, SEQ ID NO:7, SEQ ID NO:9, SEQ ID NO:11, SEQ ID NO:13, and SEQ ID NO:15.

The invention further provides for the use of an IL-17RL for the manufacture of a medicament to potentiate the activity of a bone morphogenetic protein in a mammal. The IL-17RL can be a soluble IL-17RL. The soluble IL-17RL can be selected from the group consisting of SEQ ID NO:5, SEQ ID NO:7, SEQ ID NO:9, SEQ ID NO:11, SEQ ID NO:13, and SEQ ID NO:15.

In yet another group of embodiments, the invention provides polypeptides encoded by at least one exon selected from the group consisting of exons 1 to 19 (SEQ ID NOs:55-73) of IL-17RL (SEQ ID NOs:1), which polypeptide binds to an interleukin-17 (IL-17) selected from the group consisting of IL-17A, IL-17B, IL-17C, IL-17E and IL-17F. The invention. The invention also provides nucleic acids encoding these polypeptides, and compositions comprising any of these polypeptides in a pharmaceutically acceptable carrier. Further, the invention provides the use of any of these polypeptides for the manufacture of a medicament to decrease catabolic activity in bone or cartilage.

Finally, the invention provides polypeptides with at least 85% identity to the mouse form of IL-17RL, SEQ ID NO:75, which polypeptide binds to an interleukin-17 (IL-17) selected from the group consisting of IL-17A, IL-17B, IL-17C, IL-17E and IL-17F. The invention further provides nucleic acids encoding these polypeptides, especially where the nucleic acid has the coding sequence of the mouse sequence set forth in SEQ ID NO:74. Further, the invention provides for the use of any of these polypeptides for the manufacture of a medicament and for the use of any of these nucleic acids for the manufacture of a medicament.

### DEFINITIONS

IL-17 is a pro-inflammatory cytokine that has been implicated in a number of diseases including rheumatoid arthritis (Chabaud et al., Arthritis Res, 3(3):168-77 (2001); Lubberts et al., Arthritis Rheum, 43(6):1300-6 (2000); Miossec, Curr Opin Rheumatol, 12(3):181-5 (2000)), allergic skin immune response (Albanesi et al., J Immunol, 165(3):1395-402 (2000)), organ transplant rejection (Antonysamy et al., Transplant Proc, 31(1-2):93 (1999); Antonysamy et al., J Immunol, 162(1):577-84 (1999); Loong et al., Transplant Proc, 32(7):1773 (2000); Van Kooten et al., J Am Soc Nephrol, 9(8):1526-34 (1998)), and multiple sclerosis (Matusevicius et al., Mult Scler, 5(2):101-4 (1999)). Due to the recent discovery of a family of related proteins, as discussed below, IL-17 is now also referred to as “IL-17A.”

In addition to IL-17, four related proteins have been discovered, establishing an “IL-17 family of cytokines.” These new family members, IL-17B, IL-17C, IL-17E, and IL-17F, share 20-30% homology with IL-17, and have 4 conserved cysteines and all contain a putative N-terminal signal peptide typically required for secretion (Li et al., Proc Natl Acad Sci USA, 97(2):773-8 (2000); Lee et al., J Biol Chem, 276(2):1660-4 (2001); Shi et al., J Biol Chem, 275(25):19167-76 (2000)). “IL-17F” is described in Starnes et al., J. Immunol.,167(8):4137-40 (2001) and in Hymowitz et al., EMBO J. 20(19):5332-41 (2001). As used herein, the term “IL-17” refers to any of the members of the IL-17 family of cytokines unless otherwise required by context.

“IL-17B” or “chondroleukin” are used interchangably herein to designate a member of the IL-17 family of cytokines whose full-length human sequence is given by SEQ ID NO:26. IL-17B is a secreted protein, purified from cartilage, that has catabolic activity on chondrocytes. Chondroleukin contains 8 cysteines and 1 N-glycosylation site. Computer analysis using SignalP (Nielsen et al., Protein Engineering 12(1):3-9 (1999)) predicts a signal peptide sequence of 20 amino acids, which is putatively clipped before the 160 amino acid mature protein is secreted. The secreted primary structure has a calculated molecular weight of 18.2 kDa and an estimated pI of 11. Native bovine chondroleukin runs at an apparent molecular weight of ˜23 kDa on a reducing SDS-PAGE gel. The discrepancy in molecular weight is likely due to glycosylation. Human IL-17B, or chondroleukin, is 21% identical to human IL-17. See Shi et al., supra. Chondroleukin has been designated variously in the literature as IL-17B, zcyto7, IL-20, and IL-172. See Li et al., supra; Shi et al., supra; WO 98/49310; WO 99/60127; WO 00/42188; WO 00/42189; WO 00/55204.

“IL-17 receptor like protein” and “IL-17R,” are used interchangably herein to refer to a full length protein and at least 11 splice variant proteins expressed from 19 exons of a gene on human chromosome 3p25.3-3p24.1. The longest protein expressed from these exons is encoded by the nucleotide sequence (SEQ ID NO:1) shown in. FIG. 2; the amino acid sequence (SEQ ID NO:2) is also set forth in that Figure. References herein to “full length IL-17 receptor-like protein” or “full length IL-17RL” refer to the protein of SEQ ID NO:2. SEQ ID NO:3 sets forth the nucleotide sequence encoding full length IL-17RL, but also includes the untranslated 5′ and 3′ ends shown in FIG. 2. It is noted that, due to the size of the gene, which spans some 16,500 bases (the introns alone are comprise some 14,000 bases), the genomic sequence of the gene is not set forth herein. The genomic sequence of IL-17RL and the sequence of any particular intron can, if desired, however, be determined by using the information set forth herein and in FIG. 1A.

As noted, there are at least 11 natural splice variants of full length IL-17. Six of these splice variants are truncated before the transmembrane domain and are considered to be soluble forms of the receptor. The remaining 5 variants include the transmembrane domain and are considered to be membrane bound forms of the IL-17RL. The length of the amino acid sequences of these polypeptides, and the SEQ ID NO of the nucleic acid sequence encoding the polypeptide (“NT sequence”) and the amino acid sequence of the polypeptide (“AA sequence”) are set forth in the following Table:

TABLE A

| Length of polypeptide | NT sequence | AA sequence |
|---|---|---|
| 186 amino acids | SEQ ID NO:4 | SEQ ID NO:5 |
| 269 amino acids | SEQ ID NO:6 | SEQ ID NO:7 |
| 332 amino acids | SEQ ID NO:8 | SEQ ID NO:9 |
| 348 amino acids | SEQ ID NO:10 | SEQ ID NO:11 |
| 372 amino acids | SEQ ID NO:12 | SEQ ID NO:13 |
| 409 amino acids | SEQ ID NO:14 | SEQ ID NO:15 |
| 553 amino acids | SEQ ID NO:16 | SEQ ID NO:17 |
| 683 amino acids | SEQ ID NO:18 | SEQ ID NO:19 |
| 693 amino acids | SEQ ID NO:20 | SEQ ID NO:21 |
| 703 amino acids | SEQ ID NO:22 | SEQ ID NO:23 |
| 705 amino acids | SEQ ID NO:24 | SEQ ID NO:25 |

The soluble forms of the receptor are the ones of 409 or fewer amino acids. The splice variants resulting in the polypeptides identified above are diagrammed in FIG. 1B. For example, the 409 amino acid polypeptide results from the deletion of exon 14. The start and stop of each exon is identified in FIG. 2. Splice variants other than those set forth here can be engineered, for example, by deleting different combinations of exons than those set forth in FIG. 1B, or by starting a deletion within an exon at an alternative splice junction. Such variants can be readily determined from the information set forth in FIGS. 1 and 2 and in SEQ ID NO:1 and SEQ ID NO:2 and are included herein in the use of the terms “IL-17 receptor-like protein” and “IL-17RL” unless otherwise required by context.

“Administering” a therapeutic molecule refers to processes which result in the contact of the therapeutic molecule, i.e. a polypeptide of the invention, with the cells or tissues of an organism. A therapeutic molecule may be administered by direct administration to the organism, by administering to the organism living cells that synthesize and secrete the therapeutic molecule, by administering to the organism a nucleic acid that encodes a therapeutic molecule, or by administering to the organism a molecule that stimulates the production of a therapeutic molecule.

“Bone morphogenetic protein” means a polypeptide of the TGF-β superfamily having catabolic effects on bone or cartilage. A number of bone morphogenetic proteins have been identified and include: BMP-2 (BMP-2a), BMP-3 (osteogenin), Bum (BMP-2b), BAN-5, BMP-6 (Vgr-1), BMP-7 (OP-1), BMP-8 (OP-2), BMP-9 (GDF-2), BMP-10, BMP-11, BMP-12 (GDF-11, CDMP-3), BMP-13 (GDF-6, CDMP-2) BMP-14 (GDF-5, CDMP-2), and BMP-15 (CDMP-1).

“Insoluble carrier matrix” means a biocompatible insoluble substance in which therapeutic molecules or cells may be embedded for local administration.

“Modulation,” in connection with a biological activity, refers to upregulating or downregulating the biological activity in question. For example, “modulating” catabolic effects on bone or cartilage mediated by an IL-17 cytokine refers to upregulating or downregulating the catabolic effect of the cytokine. In preferred forms, the IL-17 cytokine is IL-17B. Catabolic effects may be measured by assays such as proteoglycan synthesis and degradation by cultured chondrocytes. Effects on immune processes mediated by IL-17 cytokines, e.g., secretion of TNF-α and IL-1β by monocytes in response to IL-17B, are also indicia of modulation of the effects of a cytokine. Modulators may affect the synthesis and bioavailability of a cytokine, the interaction of a cytokine and its receptor, intracellular signaling pathways triggered by the receptor, or other biological processes.

“Chondroleukin agonist” or “IL-17B agonist” refer to a chondroleukin modulator that has the effects of chondroleukin in assays of the invention, or that potentiates the effects of chondroleukin in the assays of the invention. Chondroleukin agonists may include chondroleukin itself and chondroleukin derivatives such as truncated, modified, or mutated chondroleukin polypeptides, as well as nucleic acids that encode them. Chondroleukin agonists also include antibodies and small molecules that stimulate the chondroleukin receptor or chondroleukin signaling pathways. Generally, a chondroleukin agonist will have a specific activity 10%, 20%, 50%, 100%, 200%, 500%, or 1000% of purified chondroleukin, or will increase the biological effect of a chondroleukin polypeptide by 10%, 20%, 50%, 100%, 200%, 500%, or 1000% in assays of the invention.

“Chondroleukin antagonist” or “IL-17B antagonist” refer to a chondroleukin modulator which antagonizes a biological effect of chondroleukin in assays of the invention. Chondroleukin inhibitors include, without limitation, neutralizing polyclonal or monoclonal antibodies reactive against chondroleukin or IL-17RL, such as antibodies that sterically interfere with IL-17B interaction with IL-17RL, polypeptide fragments of chondroleukin or IL-17RL that antagonize the productive interaction of chondroleukin and the receptor, nucleic acids encoding chondroleukin inhibitor polypeptides, antisense nucleic acids directed against chondroleukin or its receptor, and other molecules that block the productive interaction of chondroleukin and its receptor or interfere with chondroleukin-induced signal transduction pathways. Chondroleukin antagonists reduce the activity of chondroleukin in the assays of the invention by 10%, 25%, 50%, 95%, 99%, or 100%.

A “patient” refers to a human or non-human mammal in need of treatment for a pathological condition. Non-human mammals include horses, cattle, goats, sheep, pigs, cats, dogs, and other domestic mamals.

“Bone or cartilage pathology” refers to a systemic or local disease, injury, or pathological condition of bone or cartilage. Such pathologies can result from either overgrowth and unwanted mineralization, or from a loss, absence, or degeneration of skeletal tissues. Bone and cartilage pathologies include degenerative disorders of bone and cartilage, defects in bone or cartilage caused by injury or disease, and congenital bone and cartilage defects.

“Degenerative cartilage disorder” means a pathological condition characterized by progressive loss of cartilage, typically articular cartilage. Degenerative cartilage disorders include osteoarthritis, rheumatoid arthritis, relapsing polychondritis and tie seronegative spondyloathropathies.

“Tissue graft” means autogenic, allogenic, or xenogenic cells introduced into the body of a mammal for therapeutic purposes. Tissue grafts may be in the form of disaggregated cells, cells embedded in a natural or synthetic carrier matrix, or cells organized into tissues. Tissue grafts may comprise a single cell type or multiple cell types mixed together.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid is separated from open reading frames that flank the gene and encode other proteins. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through translation are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occur bring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 15 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO:1 can be made detectable, e.g., by incorporating a radiolabel into the peptide, and used to detect antibodies specifically reactive with the peptide).

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” or “expression cassette” is a nucleic acid construct, generated recombinantly or synthetically, with a series of specified nucleic acid elements that permit transcription of a particular nucleic acid in a host cell. The expression vector or cassette can be part of a plasmid, virus, or nucleic acid fragment. Typically, the expression vector or cassette includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, more preferably 90%, or, in order of increasing preference, 91%, 92%, 93%, 94%, 95%, 96%, 97%, 98%, 99% or even higher identity), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length and even more preferably over the length of the amino acid sequence or coding region of a nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Nat. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

### BRIEF DESCRIPTION OF THE DRAWINGS

FIG. 1. Genomic Structure of IL-17RL. FIG. 1A. Schematic representation of the sizes of exons 1 to 19 (open boxes) and introns A to R in base pairs. Shaded area of exon 17 corresponds to the predicted transmembrane domain. FIG. 1B. Schematic diagram of exons. Lines connect exons that were joined in sequences from the EST database which represent alternative splicing events. Exons with (′) or (″) have multiple splice donor or acceptor sites evident from sequences in the EST database. FIG. 1C. Predicted sizes of translation products from alternatively spliced RNA, with grouping indicating whether the protein is predicted to be transmembrane or secreted.

FIG. 2. Annotated Nucleotide and Deduced Amino Acid Sequences of Full Length IL-17RL. Translated and untranslated regions of the cDNA are in capitalized and lower-case, respectively. The translated region of the cDNA is SEQ ID NO:1; the amino acid sequence is SEQ ID NO:2. The full nucleotide sequence, with the untranslated regions, is SEQ ID NO:3. Exons 1 to 19 (SEQ ID NOs:55-73, respectively) are indicated in numbers above the cDNA sequence. The signal peptide of the deduced protein is underlined (thin underline). Extracellular N-glycosylation sites are boxed. The transmembrane domain is underlined (thick underline). The potential sites for serine and threonine phosphorylation in the cytoplasmic domain are underlined with dashes and dots, respectively. Peptides with double-underlines were used to generate rabbit polyclonal antibodies.

FIG. 3. Expression of mRNA for the novel IL-17 receptor family member in adult human tissues. Northern blot analysis of 1 μg mRNA Multiple-Tissue Northern filters (Clontech), 2 μg mRNA isolated from primary human chondrocytes, and 5 μg total RNA isolated from either normal adult prostate or benign prostatic hyperplasia surgical specimens (BPH). Blots were hybridized with random prime [³²P] dCTP-labeled full length cDNA, washed, exposed to a phosphor-storage screen, then digitized on a Molecular Dynamics Storm system at 100 μM resolution. The 2.4 kb mRNA is indicated by the arrow.

FIG. 4. Immunoblot of homogenized human prostate electrophoresed under reducing conditions. Lane 1 was reacted with anti-ECD antibody directed against the N-terminal extracellular domain of IL17-RL. Lane 2 was reacted with anti-CYTO antibody directed against the cytoplasmic C-terminal domain of IL17-RL.

FIG. 5 Results of proteoglycan synthesis assay using primary monolayer cultures. Primary monolayer cultures of bovine articular chondrocytes in medium containing 1% fetal bovine serum (FBS) were treated with rhBMP-7 (50 ng/ml) (positive control), PBS (negative control), or several concentrations of rhCL (10 or 20 ng/ml). BMP-7 (50 ng/ml) was also used in combination with rhCL (20 ng/ml). Proteoglycan synthesis was measured by scintillation counting of incorporated ³⁵SO₄ at days 5, 8 and 10. All samples were tested in triplicate.

FIG. 6. Results of proteoglycan synthesis assay using explant cultures. Triplicate explant cultures of bovine articular chondrocytes in medium containing 0% fetal bovine serum (FBS) were treated with rhBMP-7 (50 ng/ml) (positive control), PBS (negative control), or several concentrations of rhCL (10 or 20 ng/ml). BMP-7 (50 ng/ml) was also used in combination with rhCL (20 ng/ml). Proteoglycan synthesis was measured by scintillation counting of incorporated ³⁵SO₄ at days 5 and 8.

FIG. 7. Results of proteoglycan release assay using explant cultures. Triplicate explant cultures of bovine articular chondrocytes in medium containing 0% fetal bovine serum (FBS) were treated with rhBMP-7 (50 ng/ml) (positive control), PBS (negative control), or several concentrations of rhCL (10 or 20 ng/ml). BMP-7 (50 ng/ml) was also used in combination with rhCL (20 ng/ml). Proteoglycan release was measured by scintillation counting ³⁵SO₄ in the conditioned media at days 4, 7 and 11.

### DETAILED DESCRIPTION

### Introduction

The present invention provides a new receptor, IL-17RL, that binds members of the IL-17 cytokine family, in particular, cytokine IL-17B (also known as “chondroleukin”). The invention further concerns the surprising discovery that IL-17B has catabolic effects on bone and cartilage. Hence, the invention also provides methods to reduce or ameliorate disease or injury of bone and cartilage by inhibiting chondroleukin activity, for example, by using soluble or immobilized IL-17RL or anti-IL-17B antibodies to bind IL-17B. Other diseases are marked by unwanted mineralization of tissue. The present invention also provides methods to forestall or reverse mineralization by stimulation of IL-17B activity. Further, it has been found that isoforms of IL-17RL that have epitopes on exon 6 available for binding by antibodies are present in androgen responsive prostate cancer cells, which are less invasive, but that the same epitopes are not available to antibodies as a prostate cancer loses androgen responsiveness. Therefore, the invention provides a new method of determining the aggressiveness of a prostate cancer by use of antibodies which recognize a portion of IL-17RL encoded by IL-17RL exon 6 or, more generally, the extracellular domain of IL-17RL on cells of the cancer or secreted into the surrounding medium by such cells. Moreover, androgen responsiveness can be restored to prostate cancer cells that have lost such responsiveness by introducing into the cells and expressing therein polynucleotides that encode full length IL-17RL.

A. Discovery of New Receptor for IL-17 Cytokines and Splice Variants Thereof

Surprisingly, a gene has been found that encodes a new receptor for members of the

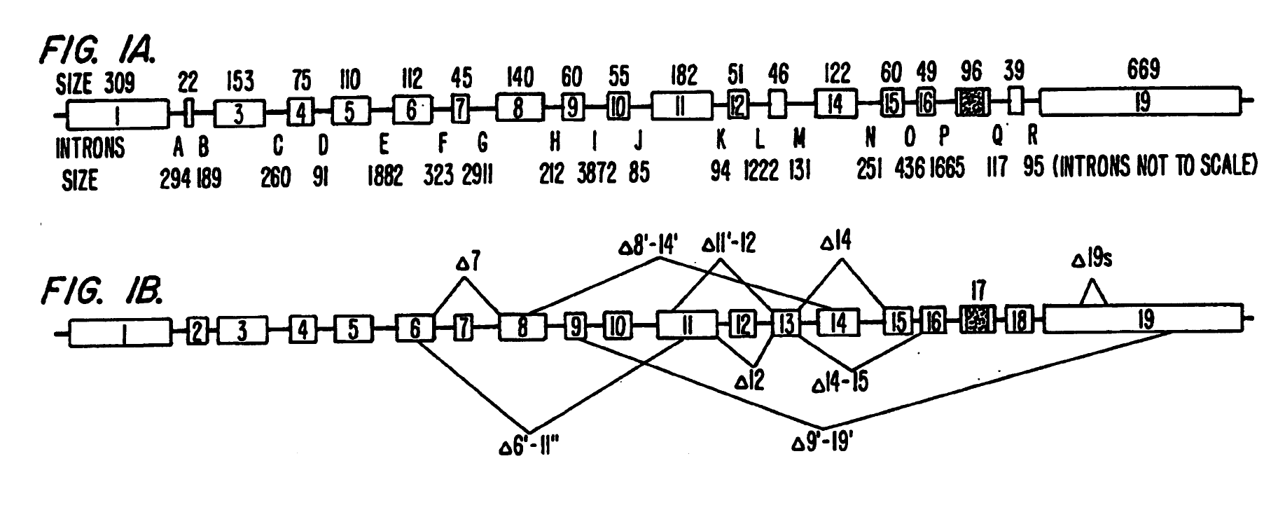

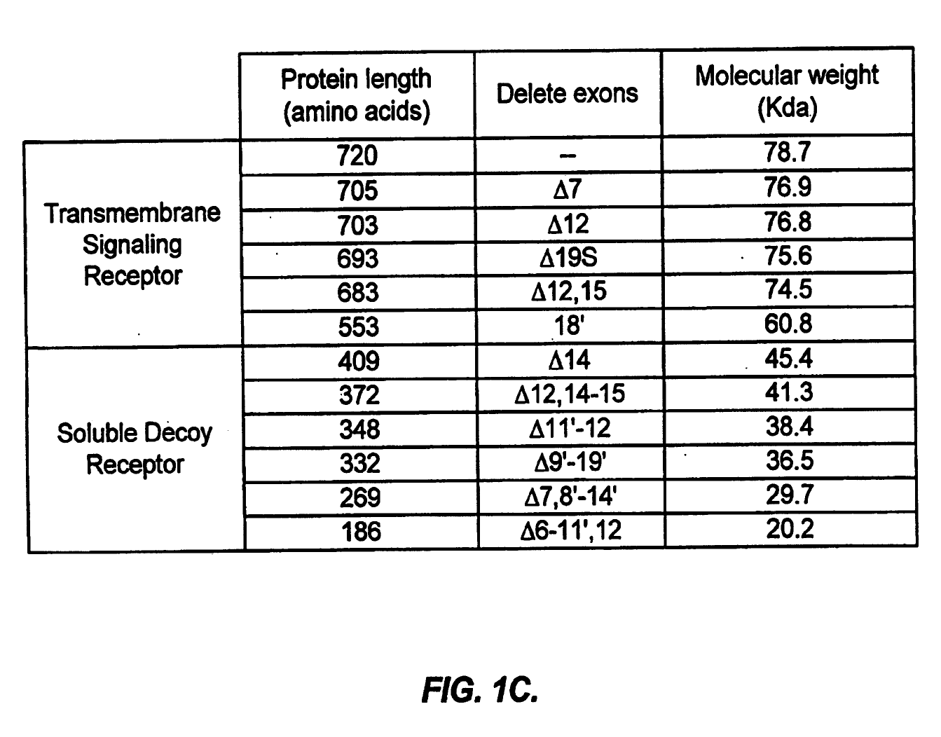

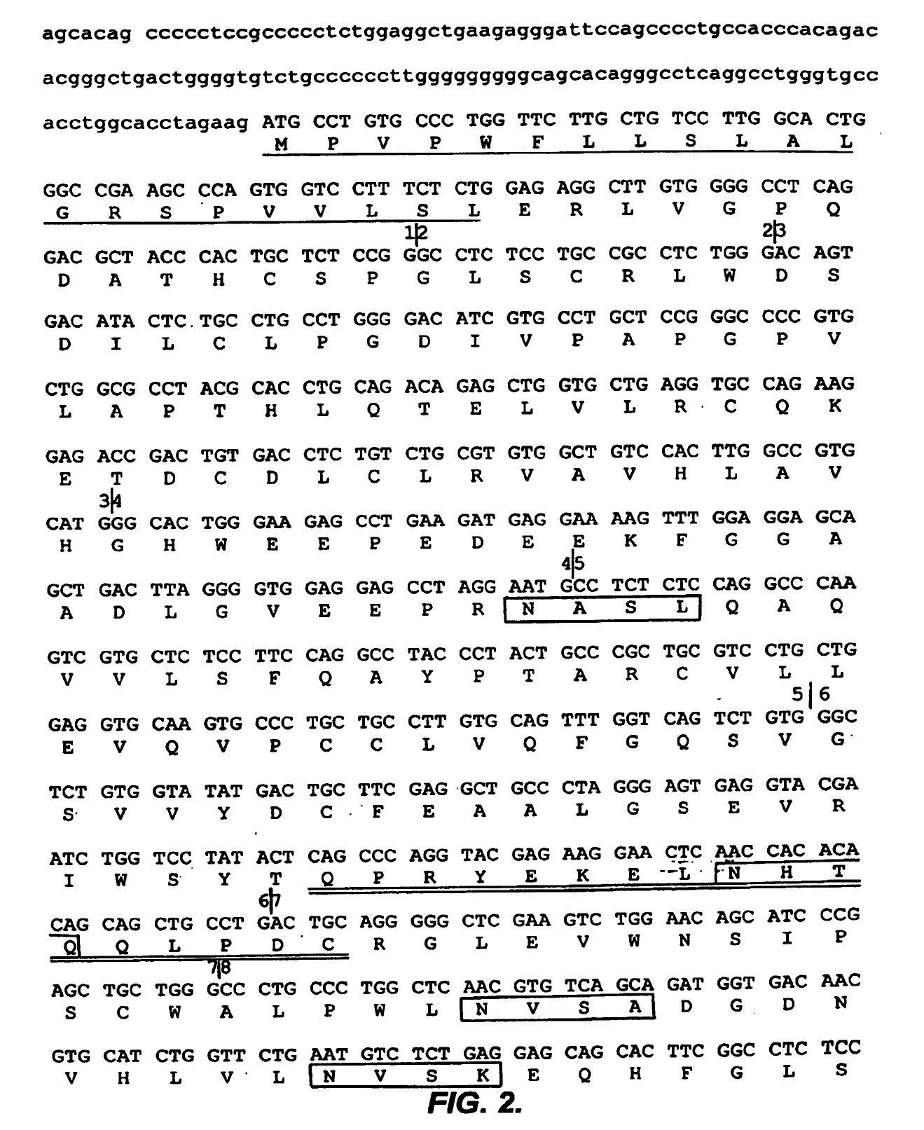

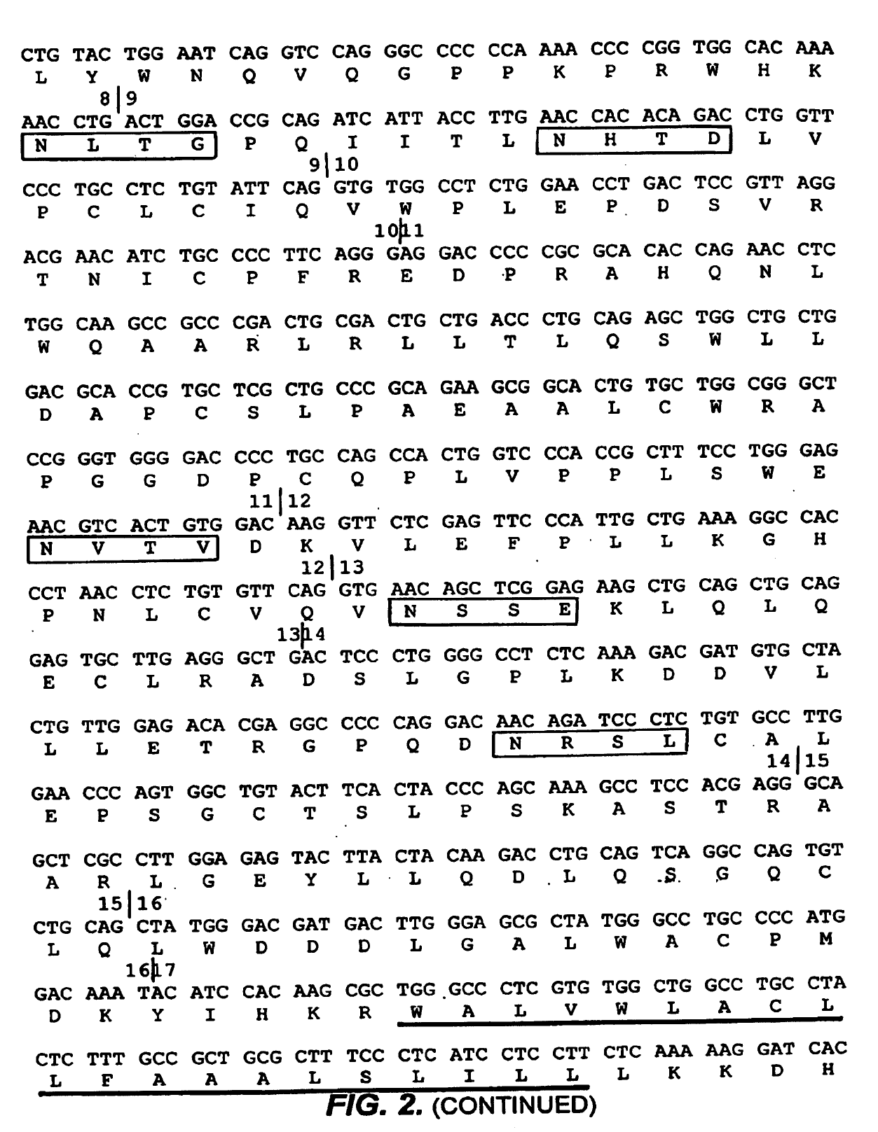

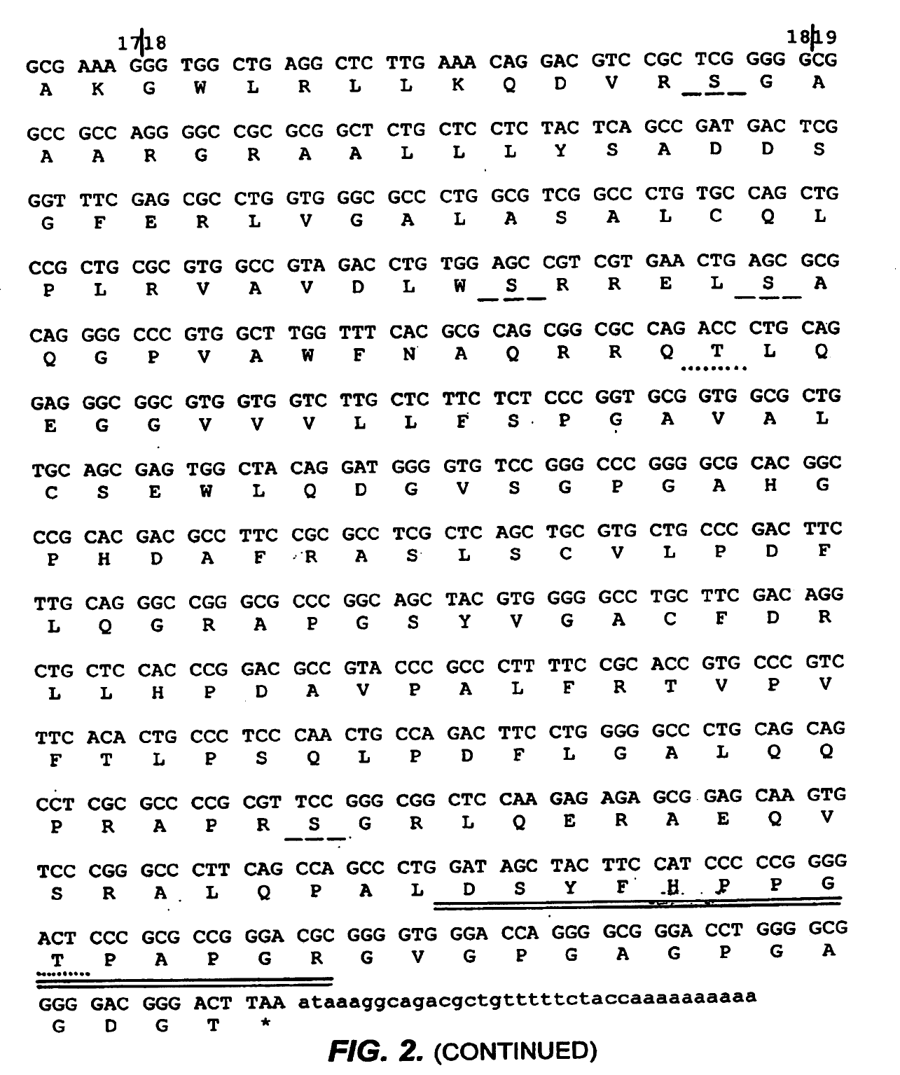

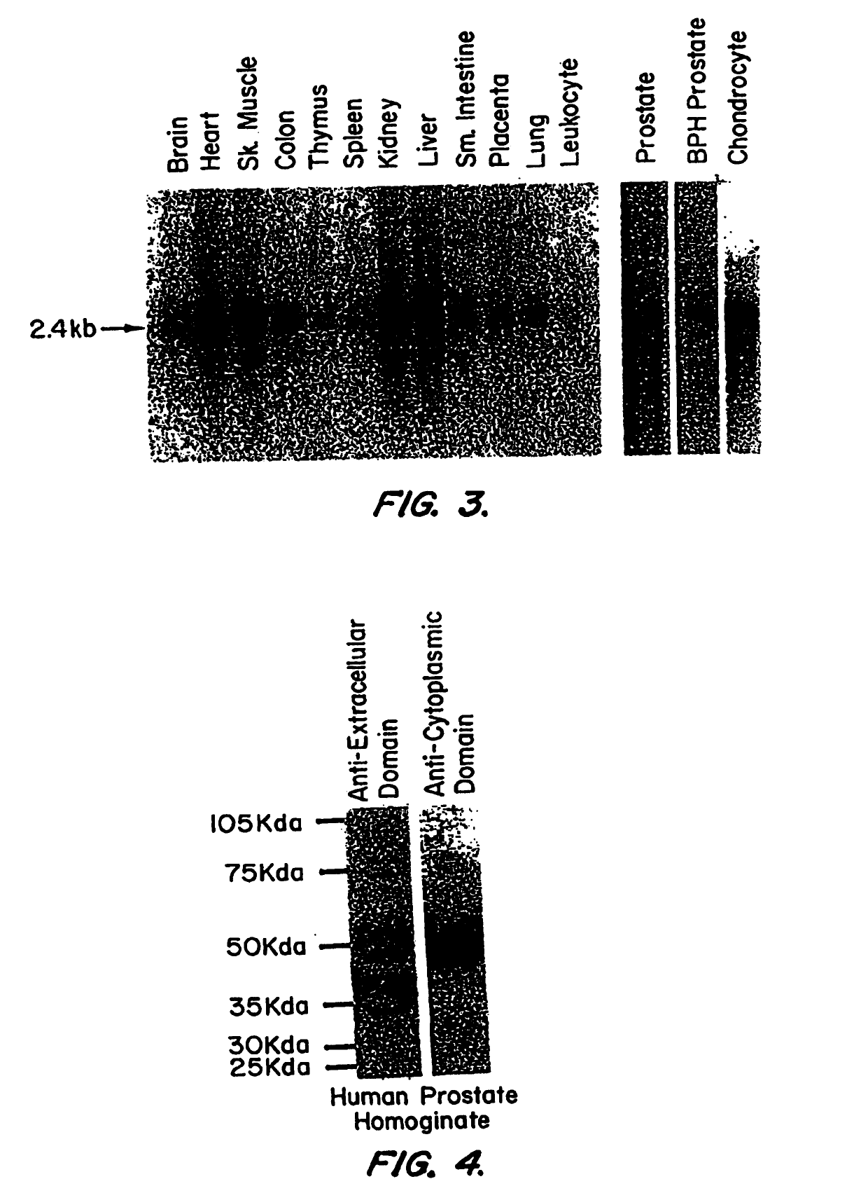

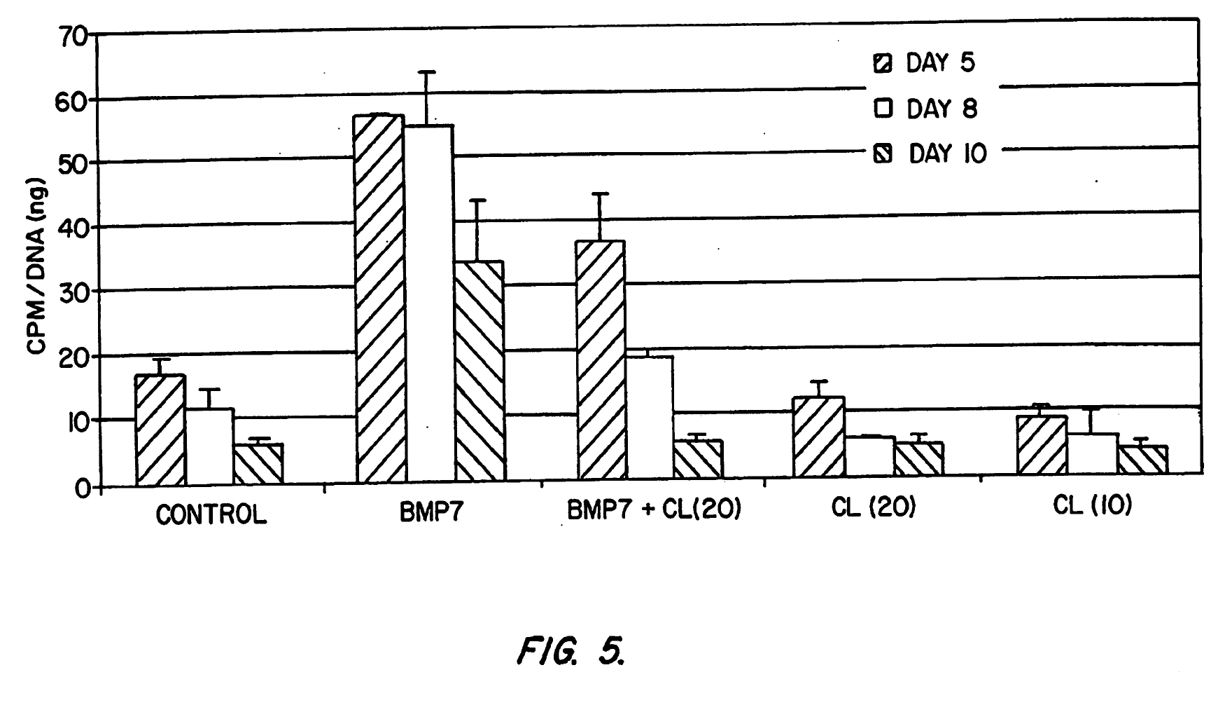

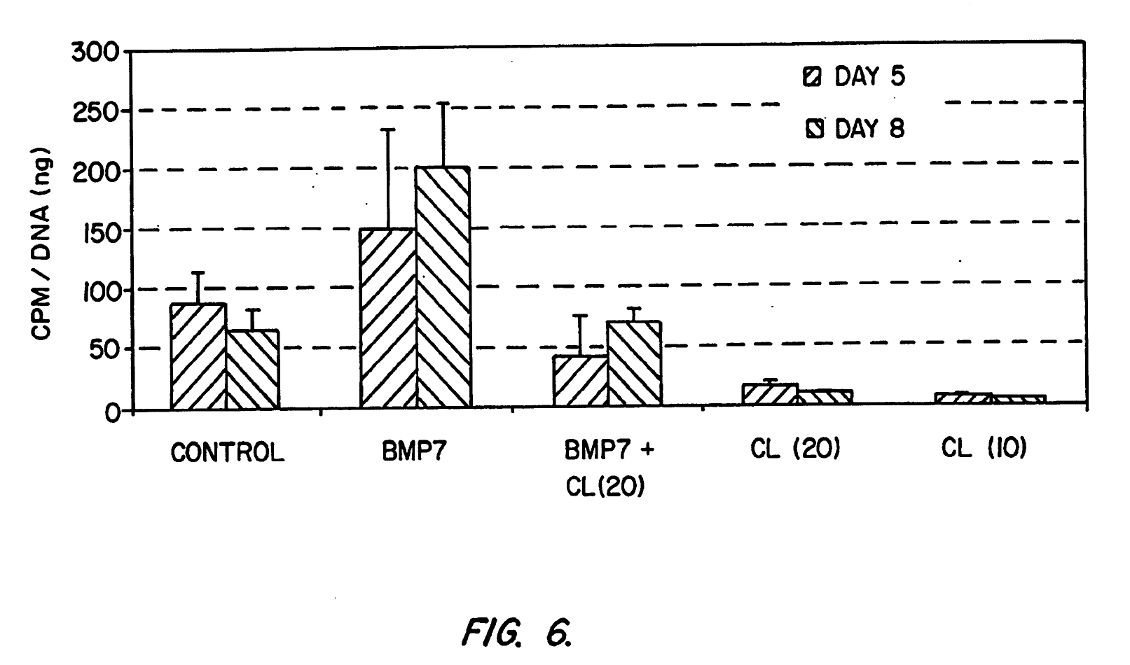

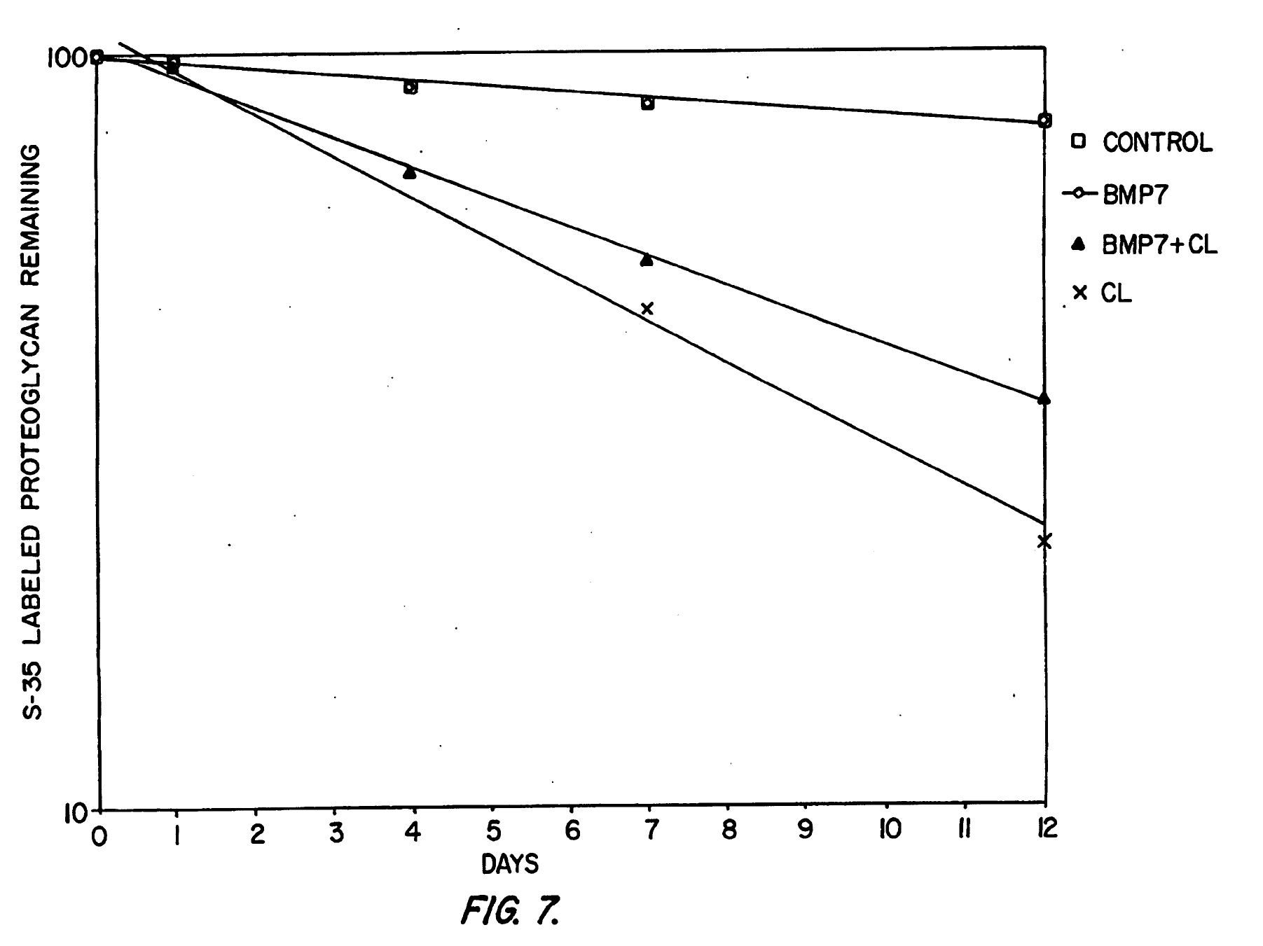

IL-17 cytokine family. In humans, the gene comprises 19 exons located on chromosome 3, and spans 16550 base pairs within the chromosomal region 3p25.3 to 3.24.1 (FIG. 1A). The full length 2402 bp cDNA (SEQ ID NO:1) of exons 1-19 is shown in FIG. 2. The translation of this open reading frame yields a 720 amino acid protein (SEQ ID NO:2), which has been designated as the IL-17 receptor-like protein, or “IL-17RL”.

Analysis of the full length amino acid sequence (SEQ ID NO:2) reveals that the initiation methionine is followed by a stretch of 20 hydrophobic amino acids with a consensus signal peptide cleavage site at position 21. The full length mature protein consists of a 447 residue amino-terminal extracellular domain, followed by a single 21 amino acid hydrophobic alpha-helical transmembrane domain encoded by exon 17, and a 232 amino acid cytoplasmic domain (see FIG. 2). The calculated molecular weight of the mature full length protein is 76,378 Daltons. The acidic extracellular domain has a predicted isoelectric point of 4.71, with nine potential sites for N-linked glycosylation. The 22 cysteines, 66 leucines and 38 prolines in the extracellular domain have the potential for forming extensive secondary structure. The 232 amino acid cytoplasmic domain contains 20 arginines, 6 histidines, and 4 lysines which contribute to its basic isoelectric point of 10.04. It also contains 16 serines, 5 threonines and 3 tyrosines. Of these, 4 serines and 2 threonines are predicted by NetPhos software to be in a context where they may be phosphorylated by intracellular kinases. There are no SH2 or SH3 domains, nor is the cytoplasmic domain predicted to have any kinase activity of its own. accession number NM_{—}014339). The cytoplasmic domains of these proteins are more conserved, sharing 25% identity and 41% similarity across their membrane-proximal 233 amino acids

Surprisingly, the 19 exons of the IL-17RL gene (exons 1-19 are SEQ ID NOs:55-73, respectively) undergo a number of alternative splicings, resulting in the natural occurrence of at least 11 shorter forms of IL-17RL. Six of these shorter IL-17RL polypeptides lack the transmembrane domain and are soluble forms of the receptor that can be introduced into a mammal as decoys to bind IL-17 cytokines, particularly IL-17B, thereby reducing the amount of IL-17 cytokines available to exert biological activity in the mammal. The following Table sets forth the length of these six polypeptides, as well as the SEQ ID NOs corresponding to the nucleotide sequence encoding the polypeptide, and the amino acid sequence of the polypeptide, respectively:

TABLE 1

| Length of polypeptide | NT sequence | AA sequence |
|---|---|---|
| 186 amino acids | SEQ ID NO:4 | SEQ ID NO:5 |
| 269 amino acids | SEQ ID NO:6 | SEQ ID NO:7 |
| 332 amino acids | SEQ ID NO:8 | SEQ ID NO:9 |
| 348 amino acids | SEQ ID NO:10 | SEQ ID NO:11 |
| 372 amino acids | SEQ ID NO:12 | SEQ ID NO:13 |
| 409 amino acids | SEQ ID NO:14 | SEQ ID NO:15 |

Five more of the splice variants contain the transmembrane domain and are considered to be membrane-bound forms of IL-17RL. The following Table sets forth the length of these polypeptides and the SEQ ID NOs corresponding to the nucleotide sequence encoding the polypeptide, and the amino acid sequence of the polypeptide, respectively:

TABLE 2

| Length of polypeptide | NT sequence | AA sequence |
|---|---|---|
| 553 amino acids | SEQ ID NO:16 | SEQ ID NO:17 |
| 683 amino acids | SEQ ID NO:18 | SEQ ID NO:19 |
| 693 amino acids | SEQ ID NO:20 | SEQ ID NO:21 |
| 703 amino acids | SEQ ID NO:22 | SEQ ID NO:23 |
| 705 amino acids | SEQ ID NO:24 | SEQ ID NO:25 |

The exon splicing that results in the splice variants set forth in Tables 1 and 2 are depicted graphically in FIG. 1B, and summarized in FIG. 1C. The start and stop sites of each exon are denoted as vertical lines in FIG. 2.

In addition to the naturally occurring splice variants set forth in Tables 1 and 2, additional variants can be engineered by standard techniques, using the information provided in FIGS. 1 and 2. The design of such variants will depend on the intended use for the polypeptide. For soluble forms of the receptor to be used as decoys for IL-17 cytokines, for example, nucleotide sequences can be designed that contain a stop codon prior to exon 17, which is the exon that contains the transmembrane domain. Examples of such nucleic acids include nucleic acids encoding any of the following polypeptides: exons 1 through and including exon 13, exon 1 through and including exon 14, exon 1 through and including exon or exon 1 through and including exon 16, or exon 1 through exon 13, and then exon 16. Conversely, if a membrane bound form of the receptor is desired, but one that lacks full signal transduction capability, the nucleic acid sequence can encode exon 1 through and including exon 18, but does not encode exon 19. The cytoplasmic domain encodes six phosphorylation sites, each of which is identified in FIG. 2. Variants of IL-17RL without or, if desired, with substantially reduced, signal transduction capability can be designed by mutating one or more of the phosphorylation sites to eliminate the serine or the threonine at the phosphorylation site or to replace them with another amino acid. Any particular construct can be tested for elimination or reduction of signal transduction capability by transfecting cells with the construct in vitro and testing them in the sulfate release assay taught in the Examples below. If both cytokine binding and full signal transduction capability are desired, it is desirable to use the full length 720 amino acid form of the receptor.

It should be noted that proteins are surprisingly tolerant of substitutions of amino acids. Many proteins have small regions in which a particular amino acid sequence is critical, and larger regions in which substitutions can be made which have limited if any impact on the protein's function. For example, enzymes typically have an active site in which a change of the amino acid sequence may significantly affect enzymatic activity, surrounded by portions of the protein in which conservative substitutions have little effect on the enzymatic activity. Thus, polypeptides can readily be designed, for example, with high degrees of identity to the polypeptides described above and which bind one or more IL-17 cytokines, but which do not have a sequence identical to those set forth above. For example, the polypeptide can have, in increasing order of preference, 85%, 86%, 87%, 88%, 89% 90%, 91%, 92%, 93%, 94%, 95%, or even higher identity to one of the polypeptide sequences of the full length or splice variant sequences of IL-17RL.

It is desirable if the variants of the receptor can bind at least one member of the IL-17 cytokine family. Thus, in some embodiments, the invention relates to polypeptides encoded by at least one exon of exons 1-19 (SEQ ID NOs:55-73) of IL-17RL which can bind an IL-17 cytokine. In general, this can be accomplished by retaining the extracellular domain of IL-17RL, which is encoded by exons 1-16 and part of exon 17. One can construct shorter forms of the extracellular domain in which portions encoded by one or more exons of the gene are deleted, so long as the resulting polypeptide is still capable of binding at least one IL-17 cytokine. Preferably, the polypeptide is encoded by at least two exons of exons 1 to 19, more preferably by three exons, still more preferably by four exons, even more preferably by five exons, yet more preferably by six, seven, eight or nine exons. Any particular polypeptide encoded by a nucleic acid constructed according to these teachings can readily be tested to confirm the ability of the polypeptide to bind members of the IL-17 family by the exemplary assays set forth in the Examples. Polypeptides that do not bind detectable amounts of at least one IL-17 cytokine selected from the group of IL-17A, IL-17B, IL-17C, IL-17D, and IL-17F are not preferred. It has previously been found that the IL-17 cytokine receptor originally designated as IL-17BR and now redesignated as IL-17Rhl binds both IL-17E and, with less affinity, IL-17B. Thus, at least some receptors for cytokines of the IL-17 family promiscuously bind more than one member of the family.

Homologs of the human receptor have been identified in several other mammals. A murine homolog of the full length protein was identified by assembly of mouse ESTs. It is 699 amino acids in length, and shares 66% identity and 75% similarity to the human protein. Two of the potentially phosphorylated serines in the cytoplasmic domain are conserved in the mouse homolog, as are 7 of the 9 N-glycosylation sites in the extracellular domain. The cDNA sequence of mouse IL-17RL, with untranslated 5′ and 3′ ends, is SEQ ID NO:74; the amino acid sequence of mouse IL-17RL is SEQ ID NO:75. Although the human forms are preferred, if desired, nucleic acid sequences derived from the nucleic acid sequence encoding mouse IL-17RL, or polypeptides derived from the mouse IL-17RL amino acid sequence can be substituted for the uses described herein for the nucleic acids and polypeptides derived from the human IL-17 sequences.

B. Discovery of Catabolic Effects of IL-17B

It has further been discovered that IL-17B, or chondroleukin, a secreted protein also identified as a cytokine, has catabolic effects on bone and cartilage. Conditions involving loss or injury of bone and cartilage are widespread, and their pathology involves an imbalance between anabolic and catabolic activities. Hence, the invention provides methods to reduce or ameliorate disease or injury of bone and cartilage by inhibiting chondroleukin activity. Other diseases are marked by unwanted mineralization of tissue. The present invention provides methods to forestall or reverse mineralization by stimulation chondroleukin activity. Accordingly, a variety of chondroleukin agonists and antagonists are useful in the practice of the invention. General methods for identifying chondroleukin modulators, as well as specific chondroleukin modulator molecules, may be found in PCT publications WO 98/49310, WO 99/60127, WO 00/42189, and WO 00/55204.

C. Uses of IL-17RL

IL-17RL has a variety of in vivo and in vitro uses. As discussed in more detail below, IL-17 cytokines and, in particular, IL-17B are found in arthritic cartilage and cause cartilage degradation. In one set of embodiments, the presence of IL-17RL is used to monitor progression of osteoarthritis in a patient. Osteoarthritis is a generally age-dependent form of the disease that accounts for some 70% of all arthritis and is the most common joint disease. IL-17RL exists in both membrane bound and secreted forms. Samples of cells from within the affected joint and of the synovial fluid are taken and the ratio of IL-17RL on the cell surface to levels in the synovial fluid is determined. An increase in the ratio over time indicates a higher level of the secreted form of the receptor, which is associated with a more advanced form of the disease. The practitioner can then use higher levels of painkillers and anti-inflammatory agents to help ease discomfort. In another set of embodiments, IL-17RL is used in vivo to decrease the catabolic effects of IL-17B and other members of the IL-17 cytokine family by being administered as a decoy, thereby preventing the IL-17B or another IL-17 cytokine family member from reaching endogenous IL-17RL on cells. In this use, the administered IL-17RL decreases the free IL-17 cytokine available to interact with the natural IL-17RL receptor by competitive inhibition, thereby reducing IL-17 cytokine mediated or induced degradation.

Typically, in these uses, IL-17RL is administered to reduce or alleviate osteoarthritis. In these cases, the IL-17RL is administered by injection into the synovial fluid surrounding a joint by direct intra-joint injection or during arthroscopic examination. The administration can be a one-time injection, or a series of injections. For longer term reduction of catabolic activity in a joint due to IL-17 cytokines, the IL-17RL can be embedded in an insoluble gel matrix shaped to fit in the joint, which is then implanted during arthroscopic or open-field surgery. The matrix then secretes the IL-17RL over a period of time. Alternatively, IL-17RL can be immobilized on the matrix or another solid support by standard conjugation techniques to bind IL-17B to the matrix or other support, reducing the amount available to bind to endogenous receptors. In some uses, nucleic acids encoding soluble forms of IL-17RL, or forms of IL-17RL comprising the transmembrane but lacking signal transduction capability, are introduced into synovial cells or other cells in a joint. Expression of the IL-17RL then acts as a decoy for IL-17 cytokines, such as IL-17B, decreasing the amount available to bind to endogenous IL-17RL.

As noted above, the previously discovered IL-17Rhl receptor binds two members of the IL-17 cytokine family, IL-17E and IL-17B, although it binds IL-17B with less affinity than it does IL-17E. Therefore, higher concentrations of IL-17Rhl are required to bind a given amount of IL-17B than to bind a like amount of IL-17E. While IL-17RL is preferentially used to bind IL-17B, it can also be used to bind other IL-17 cytokines. The amount of IL-17RL to administer will therefore desirably be adjusted depending on the particular member of the IL-17 cytokine family one wishes to bind. The relative amounts to use for any given IL-17 cytokine can be readily determined using the assays set forth herein.

It has also been discovered that IL-17RL can be used as a marker for the aggressiveness of prostate carcinoma. Bone morphogenetic proteins (BMPs) are key players in the ability of prostate cancer to metastasize to bone. Less aggressive (metastatic) prostate carcinoma tends to be androgen-responsive; while loss of androgen responsiveness is associated with increased aggressiveness and metastatic potential. In the United States, the most common system of grading the aggressiveness of prostate cancer is the Gleason system, in which sections of tissue are reviewed. Typically, the pathologist looks at the “architecture” of the sample and assigns a first and a second Gleason grade, each of which is from 1 to 5. The two are then added to form a Gleason score. A higher Gleason score is associated with a poorer outcome for the patient than a lower Gleason score.

It has now been discovered that IL-17RL is expressed in normal prostate epithelium and can be detected by antibodies directed to portions of the extracellular domain of the IL-17RL protein, such as that encoded by IL-17RL exon 6. It has further been found that the receptor is either lost, or that portions of the extracellular domain become unavailable to antibodies in prostate carcinoma as the cancer becomes more aggressive and loses androgen-dependence. The great majority of prostate cancers are carcinomas, or cancers of epithelial cells. In normal prostate, the epithelial cells immediately surrounding the lumens in the prostate are seen to be labeled with antibodies directed to an IL-17RL extracellular epitope, while the connective tissue cells known as mesenchyme were labeled more lightly or equally. In higher Gleason scored carcinoma, however, it was noted that the connective tissue cells were more strongly labeled than the epithelial cells, which appear to have lost labeling.

Thus, a sample of a prostate carcinoma can be examined by immunohistochemistry to determine whether extracellular epitopes such as that encoded by IL-17RL exon 6 are present. In this regard, a tumor can be sectioned and a thin section contacted with anti-IL-17RL antibodies under conditions which permit the binding of the antibodies to be detected (for example, the antibodies can be labeled, detected by the addition of a second antibody which binds to the first, or by other conventional immunological techniques). Exemplary immunohistochemical studies are reported in the Examples, below. Higher grade cancers tended to show decreased reactivity in epithelial cells and an increased reactivity in stromal regions, relative to normal prostate. If IL-17RL is not detected, its absence indicates that the cancer has lost responsiveness to androgen and is more aggressive. The practitioner can then take this information into account in planning a therapeutic regimen for the patient.

### ASSAYS FOR IDENTIFYING IL-17B MODULATORS

Modulators of IL-17B activity may be identified by a variety of assay procedures known in the art. For example, modulator molecules may be identified initially by their ability to bind to chondroleukin or to IL-17RL, and to block or promote the physical interaction between chondroleukin and the receptor. Biological assays that measure the ability of a compound to modulate biological responses induced by chondroleukin are typically used to further investigate such compounds, although biological assays may also be employed to initially identify IL-17B modulators.

One assay for IL-17B modulators is their ability to modulate IL-17B -induced immunological responses. Purified chondroleukin induces neutrophil migration when injected into the peritoneal cavity of mice (Shi et al., J Biol Chem 275(25):19167-76 (2000)) and stimulates the secretion of TNF-α and IL-1β from monocyte cell lines in vitro. (Li et al., Proc Nat Acad Sci USA 97(2):773-8 (2000)). Thus, chondroleukin modulators may be screened by their ability to block or promote chondroleukin-induced neutrophil migration or cytokine secretion.

Agonists and antagonists may also be assayed for their ability to modulate the effects of chondroleukin on skeletal tissues, and their ability to modulate chondroleukin's antagonism of By activity. By taking advantage of the discovery that chondroleukin is catabolic for cartilage and opposes BMP activity, agonists and antagonists of the invention may be assayed for their specific effects on matrix metabolism or BMT activity. Any assay system measuring the dynamic properties of bone and cartilage may be used to assay chondroleukin modulators. Preferred assays are the chondrocyte proteoglycan metabolism assays described in the Examples. Incorporation of radiolabeled proteoglycan precursors into adherent material measures the rate of cartilage matrix synthesis, while release of radiolabeled precursors into the culture medium measure the rate of cartilage matrix degradation. Since chondroleukin decreases the rate of matrix synthesis and increases the rate of matrix degradation, chondroleukin agonists and antagonists may be identified by their ability to enhance or retard the effects of chondroleukin on matrix synthesis or degradation. Chondrocyte metabolism may be assayed either with explants of articular cartilage, or with dissociated chondrocyte cultures as described in the Examples. To identify agents that modulate chondroleukin's antagonism of BMP activity, recombinant BMPs may be included in the chondrocyte assay systems of the Examples. Alternatively, the effects of chondroleukin modulators to enhance or attenuate BMP function may be assayed in standard BMP systems known in the art, such as induction of cartilage at ectopic sites (Luyten et al., J Biol Chem 264(23):13377-80 (1989)) or in vitro (Lietman et al., J Bone Joint Surg Am 79(8): 1132-7 (1997)).

The in vitro assays described in the Examples and known in the art are useful for high-throughput screening procedures, e.g., when selecting chondroleukin modulators from a combinatorial library of compounds. However, where the most realistic simulation of the in vivo milieu is desirable, the in vivo chondrogenic activity assay of the Examples may be employed. A chondroleukin modulator is precipitated, along with a BMP or other chondrogenic or osteogenic factor, onto an insoluble carrier matrix as described in the Examples. Following subcutaneous implantation in a rat or other test animal, the extent of cartilage formation within and around the insoluble matrix measures the chondrogenic activity of the test substances embedded in the matrix. Typical dosage of a BMP or chondroleukin modulator is 50-1000 ng for a rat-sized animal, and 1 μg-10 mg for a human-sized animal. Pellets lacking chondrogenic activity are colonized by fibroblast-like cells; in pellets with chondrogenic activity these or other stem-like cells differentiate to chondrocytes within the pellet. A saturating level of BMP serves as a positive control.

Typically, pellets are removed after 11 days, fixed and sectioned, and stained with toluidine blue. Chondrogenic activity may be assessed and assigned a score of 0-4 by qualitative evaluation of the staining intensity. Barely detectable staining around one or two chondrocytes receives a score of 1, while extensive staining of matrix surrounding many colonies of chondrocytes receives a score of 4. Intermediate staining patterns receive scores of 2 or 3.

Although chondrogenic pellets will begin to ossify at 11 days, ossification being complete in 20-30 days, cartilage developing in a chondrogenic pellet begins to express alkaline phosphatase activity following 9 days of implantation. Hence, chondrogenesis may be more precisely quantitated by assaying a portion of the explanted pellet for alkaline phosphatase activity. Alkaline phosphatase activity is normalized to the DNA content of the sample to control for the number of cells assayed.

Little or no cartilage formation is induced by guanidine-extracted collagenous bone matrix alone. However, adding BMPs (50 ng-1000 ng) or protein extracts of skeletal tissue renders the matrix chondrogenic. Exogenous chondroleukin inhibits the observed chondrogenic activity. Hence, chondroleukin agonists will decrease cartilage deposition by antagonizing the catabolic activity of BMPs Likewise, chondroleukin antagonists will neutralize the inhibitory effect of endogenous chondroleukin in the test animal, and increase the extent of matrix deposition in the pellet.

While the in vivo chondrogenic assay system may be employed to confirm the activity of a chondroleukin modulator identified by other means, it may also be used to determine the optimal dosage of a chondroleukin modulator in a therapeutic context. For example, to determine the dosage of a chondroleukin inhibitor needed to relieve chondroleukin's effect on a BMP, a series of test pellets impregnated with a fixed amount of BMP and a varying amount of chondroleukin inhibitor may be implanted in a rat as described in the Examples. The optimum dosage of chondroleukin inhibitor is the dosage at which maximum cartilage formation is observed. By simply implanting the test pellet in the appropriate anatomical location, the optimum dosage of a chondroleukin inhibitor may be determined without measuring the endogenous level of chondroleukin itself Alternatively, the endogenous level of chondroleukin in a location of interest may be determined by immunoassay, and the test pellet may be impregnated with sufficient recombinant chondroleukin to mimic the location of interest when the pellet is implanted subcutaneously.

### EXPRESSION AND PURIFICATION OF POLYPEPTIDE MODULATORS

Chondroleukin modulators may be recombinant polypeptides, e.g., soluble receptors, fragments of chondroleukin or its receptor, and engineered antibodies. Recombinant polypeptide modulators are most easily obtained by recombinant DNA methodology. To obtain high level expression of a cloned gene, such as those cDNAs encoding polypeptide chondroleukin modulators, one typically subclones the cDNA into an expression vector that contains a strong promoter to direct transcription, a transcription/translation terminator, and a ribosome binding site for translational initiation. Suitable bacterial promoters are well known in the art and described, e.g., in Sambrook et al. and Ausubel et al. Bacterial expression systems for expressing polypeptide chondroleukin modulators are available in, e.g., E. coli, Bacillus sp., and Salmonella (Palva et al., Gene 22:229-235 (1983); Mosbach et al, Nature 302:543-545 (1983). Kits for such expression systems are commercially available. Especially preferred are bacterial expression systems offering high level, tightly regulated expression, such as the pET series of vectors and compatible expression hosts available from Novagen (pET System Manual, Novagen, Madison, Wis.). Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the polypeptide chondroleukin modulator encoding nucleic acid in host cells. A typical expression cassette thus contains a promoter operably linked to the nucleic acid sequence encoding a polypeptide chondroleukin modulator, ribosome binding sites, transcription or translation termination signals, and, in cassettes adapted to eukaryotic hosts, signals required for efficient polyadenylation of the transcript, Additional elements of a eukaryotic expression cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a polypeptide chondroleukin modulator encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of polypeptide chondroleukin modulators, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing polypeptide chondroleukin modulators.

After the expression vector is introduced into the cells, the transfected cells are cultured under conditions favoring expression of polypeptide chondroleukin modulators, which is recovered from the culture using standard techniques identified below.

Purification of Polypeptide Chondroleukin Modulators

Either naturally occurring or recombinant polypeptide IL-17B modulators can be purified for use in the methods and compositions of the invention. Polypeptide IL-17B modulators may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptide chondroleukin modulators are being purified. For example, proteins having established molecular adhesion properties can be reversibly fused to the polypeptide chondroleukin modulators. With the appropriate ligand, the polypeptide chondroleukin modulators can be selectively adsorbed to a purification column and then freed from the column in a relatively pure form. The fused protein is then removed by enzymatic activity. Finally the polypeptide chondroleukin modulators may be purified using immunoaffinity columns.

A. Purification of Polypeptide Chondroleukin Modulators from Recombinant Bacteria

Recombinant proteins are expressed by transformed bacteria in large amounts, typically after promoter induction; but expression can be constitutive. Promoter induction with IPTG is a one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of the polypeptide chondroleukin modulators from inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Polypeptide chondroleukin modulators proteins are separated from other bacterial proteins by standard separation techniques, e.g., with Ni-NTA agarose resin in the case of polypeptide chondroleukin modulators comprising a metal-chelating moiety.

Alternatively, it is possible to purify the polypeptide chondroleukin modulators from bacterial periplasm. After lysis of the bacteria, when the polypeptide chondroleukin modulators are exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying the Polypeptide Chondroleukin Modulators

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30% of saturation. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the polypeptide chondroleukin modulators can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The polypeptide chondroleukin modulators can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### CHONDROLEUKIN MODULATORS

Soluble Receptors

Soluble chrondroleukin receptors (sCLR) are useful as IL-17B antagonists. Soluble forms of IL-17RL are particularly preferred IL-17B antagonists in the methods of the invention.

Natural or synthetic soluble receptors compete with cell surface receptors for binding of extracellular signaling molecules. In general, synthetic recombinant soluble receptors comprise the extracellular domain, but not the transmembrane or cytoplasmic domains, of membrane-bound receptors. By reducing the number of signaling molecules that bind to membrane-bound receptors, soluble receptors downregulate biological responses to extracellular signaling molecules. In particular, soluble forms of cytokine receptors can inhibit cytokine response by competition with membrane-bound cytokine receptors. Soluble cytokine receptors have been effective in treating autoimmune or inflammatory conditions, such as rheumatoid arthritis, where cytokines are mediators of tissue damage. See Fernandez-Botran, Expert Opin Investig Drugs 9(3):497-514 (2000). In particular, U.S. Pat. Nos. 6,083,906 and 6,100,234 describe recombinant soluble IL-17 receptors that inhibit IL-17 mediated signaling, and the use of soluble IL-17 receptors to reverse excessive nitric oxide (“NO”) production in osteoarthritic cartilage.

Recombinant soluble IL-17B receptors, such as soluble forms of L-17RL, may be administered or expressed in tissues to reduce cartilage catabolism, or to potentiate BMPs. These soluble receptors may be produced in any of the prokaryotic or eukaryotic expression systems known in the art, and the ability of the receptors to block IL-17B activity may be assayed in any of the assay systems of the invention described herein.

Antibodies

Antibodies to inflammatory cytokines or their receptors are clinically effective agents in treating cartilage disorders. See, e.g., Maini et al., Annu Rev Med 51(1):207-29 (2000). Accordingly, antibodies against IL-17B or that bind to the extracellular domain of the IL-17B receptor (thereby blocking binding of IL-17B and downregulating its biological effects) may be employed as IL-17B antagonists. Antibodies that bind to the transmembrane or cytoplasmic domains of IL-17RL can be used to detect the presence of IL-17RL in a biological sample, such as a prostate cancer biopsy. Some antibodies directed against IL-17B will instead act as IL-17B agonists, by stimulating IL-17RL in the absence of an IL-17 cytokine. Antibodies that act as agonists and antagonists to IL-17Rhl, another member of the IL-17 cytokine family, are described in WO 98/49310, WO 99/60127, WO 00/42189, and WO 00/55204.

Generation of polyclonal antibodies against IL-17B and the extracellular domain of its receptor is described in the Examples. However, in a preferred therapeutic embodiment, agonist or antagonist antibodies against chondroleukin or its receptor are monoclonal antibodies. Generation of monoclonal antibodies is well known in the art and typically employs hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes nay be immunized in vitro. The immunizing agent will typically include an IL-17B or to IL-17RL polypeptide, or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens or that have binding specificities for two epitopes on the same antigen. Alternatively, tetramer-type technology may create multivalent reagents.

In a preferred embodiment, the antibodies to IL-17B or to IL-17RL are capable of reducing or eliminating a biological function of chondroleukin, as is described below. That is, the addition of IL-17B or to IL-17RL antibodies (either polyclonal or preferably monoclonal) to a tissue or cells will reduce or eliminate the catabolic and anti-BMP effects of chondroleukin. Generally, at least a 25% decrease in activity is preferred, with at least about 50% being particularly preferred and about a 95-100% decrease being especially preferred.

In a preferred embodiment the antibodies to IL-17B or to IL-17RL are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Polynucleotide Modulators

In certain embodiments, the activity of IL-17B or IL-17RL can be downregulated, or entirely inhibited, by the use of antisense polynucleotide, i.e., a nucleic acid complementary to, and which can preferably hybridize specifically to, a coding mRNA nucleic acid sequence, e.g., an IL-17B or IL-17RL mRNA, or a subsequence thereof. Binding of the antisense polynucleotide to the mRNA reduces the translation and/or stability of the mRNA. Conversely, chondroleukin activity may be upregulated by transforming cells with polynucleotides that encode IL-17B or IL-17RL, or a regulatory protein that increases expression of IL-17B or IL-17RL.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing is species which are known for use in the art. Analogs are comprehended by this invention so long as they function effectively to hybridize with IL-17B or IL-17RL mRNA. Such polynucleotides and analogs are manufactured by, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for chondroleukin or its receptor. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

In addition to antisense polynucleotides, ribozymes can be used to target and inhibit transcription of IL-17B or IL-17RL nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al. (1994) Adv. in Pharmacology 25: 289-317 for a general review of the properties of different ribozymes).

The general features of harpin ribozymes are described, e.g., in Hampel et al. (1990) Nucl. Acids Res. 18: 299-304; Hampel et al. (1990) European Patent Publication No. 0 360 257; U.S. Pat. No. 5,254,678. Methods of preparing are well known to those of skill in the art (see, e.g., Wong-Staal et al., WO 94/26877; Ojwang et al. (1993) Proc. Natl. Acad. Sci. USA 90: 6340-6344; Yamada et al. (1994) Human Gene Therapy 1: 39-45; Leavitt et al. (1995) Proc. Natl. Acad. Sci. USA 92: 699-703; Leavitt et al. (1994) Human Gene Therapy 5: 1151-120; and Yamada et al. (1994) Virology 205: 121-126).

Polynucleotide modulators of IL-17B or IL-17RL may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of IL-17B or IL-17RL may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment. In preferred embodiments, the polynucleotide introduced encodes full length IL-17RL. In one especially preferred embodiment, the polynucleotide construct is introduced into prostate cancer cells which have lost androgen-responsiveness to restore androgen-responsiveness to the cells, thereby reducing their aggresiveness. Such introduction is especially useful for tumors that cannot be conveniently be surgically removed or ablated, particularly for prostate metastases of the patient's bones.

Small Molecules and Protein Fragments

In certain embodiments, combinatorial libraries of potential modulators will be screened for an ability to enhance or inhibit chondroleukin activity. These modulators may affect the interaction of IL-17 cytokines and IL-17RL, or may perturb intracellular signaling networks involved in the response to IL-17B. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al. (1994) J. Med. Chem. 37(9): 1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88), peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Stratagene, Corp.), peptide nucleic acid libraries (see, e.g., U.S. Pat. No. 5,539,083), antibody libraries (see, e.g., Vaughn et al. (1996) Nature Biotechnology, 14(3): 309-314), and PCT/US96/10287), carbohydrate libraries (see, e.g., Liang et al., (1996) Science, 274: 1520-1522, and U.S. Pat. No. 5,593,853), and small organic molecule libraries (see, e.g. benzodiazepines, Baum (1993) C&EN, January 18, page 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.).

The assays to identify modulators are amenable to high throughput screening. Preferred assays thus detect modulation of chondroleukin biological activity, e.g., its catabolic activity on chondrocytes. However, modulators may also be screened on the basis of their ability to upregulate or downregulate the synthesis of chondroleukin or chondroleukin receptor nucleic acids and polypeptides.

High throughput assays for the presence, absence, quantification, or other properties of particular nucleic acids or protein products are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Most preferred are fragments of chondroleukin itself. Chondroleukin fragments may act as agonists analogous to chondroleukin. However, some chondroleukin fragments will act as competitive inhibitors of the productive interaction between chondroleukin and its receptor.

In some embodiments, modulators are peptides of from about 5 to about 30 amino acids, with from about 5 to about 20 amino acids being preferred, and from about 7 to about being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

### METHODS OF THERAPEUTIC ADMINISTRATION

Gene Therapy

The IL-17B agonists and antagonists of the invention may be administered to patients directly in their active form. For small molecule agonists and antagonists, direct administration of the molecule may be necessary. Protein or polynucleotide modulators, however, may be administered directly or as polynucleotides encoding the modulator. Administration of various therapeutic genes has proven safe and effective to protect cartilage from degradation, and to repair cartilage that has become damaged as a result of disease or injury. See Evans et al., Clin Orthop 3:S214-9 (2000). IL-17B's ability to degrade cartilage indicates that IL-17B inhibitors have chondroprotective or chondroregenerative effects. Accordingly, polynucleotides encoding IL-17B antagonists, such as IL-17RL, may be employed as chondroprotective and chondroregenerative medicines in the same way that, and in combination with, antagonists of inflammatory cytokines are employed in the art to treat cartilage injury and disease. For many applications, expression of IL-17B modulators for a relatively short time (e.g., 12, 30, or 60 days) will suffice to repair the damaged bone or cartilage. Hence, the invention may be practiced with gene therapy systems in which expression of the therapeutic polynucleotides is transient.

Some conditions for which IL-17B agonists and antagonists are useful are systemic conditions—e.g., Paget's disease or atherosclerosis. Systemic administration of polynucleotides encoding IL-17B modulators is suitable to treat such conditions. In other embodiments, including cartilage regeneration or injury repair, gene therapy may be localized. Localized gene therapy may be practiced either by local administration of polynucleotides encoding chondroleukin modulators, or by local administration of cells transformed ex vivo.

In ex vivo gene therapy, cells from the patient may be removed, transfected with the therapeutic polynucleotide, expanded in culture, and re-administered to the site of injury or disease. Allogenic or xenogenic cells may also be employed, taking into account histocompatibility issues known in the art. Removal, expansion, and re-administration of chondrogenic cells is an effective procedure for treatment of cartilage defects. See Brittberg, in Grifka & Ogilvie-Harris eds., Osteoarthritis: Fundamentals and Strategies for Joint-Preserving Treatment, Springer-Verlag (2000). Hence, isolation, culture, and transformation of chondrocytes with polynucleotides encoding chondroleukin modulators, followed by transplantation to the patient, is a suitable method for ex vivo gene therapy. Ex vivo transformation of synoviocytes and mesenchymal stem cells with therapeutic genes has also been successful in the treatment of cartilage injury (see Evans et al., Clin Orthop 3:S214-9 (2000)), and synoviocytes and mesenchymal stem cells are also suitable for transformation with polynucleotides encoding chondroleukin modulators. While transformation of dissociated cells in culture is a preferred method for administering polynucleotides encoding chondroleukin modulators, organized tissues such as osteochondrum or periosteum may also be transformed during transplantation procedures. Combinations of therapies are also within the scope of the invention, as polynucleotides encoding chondroleukin modulators may be administered as adjunct therapeutics in the course of transplantation therapy or other tissue engineering procedures.

Nucleic acids encoding IL-17B modulators can be inserted into any of a number of well-known vectors for the transfection of target cells and organisms as described below. The nucleic acids are transfected into cells, ex vivo or in vivo, through the interaction of the vector and the target cell. The nucleic acid for a chondroleukin modulator, under the control of a promoter, then expresses a chondroleukin modulator of the present invention, thereby enhancing or inhibiting the biological effects of chondroleukin. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiologic and Immunology (Doerfler & Böhm eds., 1995); and Yu et al., Gene Therapy 1:13-26 (1994)).

Delivery of the gene or genetic material into the cell is the first step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahrad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in video) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin et al., Mol. Cell. Biol. 4:2072-2081 (1984); Hermonat & Muzyczka, Proc. Natl. Acad. Sci. U.S.A. 81:6466-6470 (1984); and Samulski et al., J. Virol. 63:03822-3828 (1989).

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors (Ellem et al., Immunol Immunother. 44(1):10-20 (1997); Dranoff et al., Hum. Gene Ther. 1:111-2 (1997)).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998), Kearns et al., Gene Ther. 9:748-55 (1996)).

Replication-deficient recombinant adenoviral vectors (Ad) are predominantly used transient expression gene therapy, because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 241:5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Administration is by any of the routes normally used for introducing a molecule into ultimate contact with blood or tissue cells. The nucleic acids are administered in any suitable manner, preferably with pharmaceutically acceptable carriers. Suitable methods of administering such nucleic acids are available and well known to those of skill in the art, and, although more than one route can be used to administer a particular composition, a particular route can often provide a more immediate and more effective reaction than another route.

Administration Routes

Systemic administration of IL-17B modulators is achieved by pharmaceutical compositions and techniques well known in the art. There are a wide variety of suitable pharmaceutically acceptable excipients that can be used to fashion compositions of the present invention (see, e.g., Remington's Pharmaceutical Sciences, 17^{th} ed., 1989). Administration can be in any convenient manner, e.g., by injection, oral administration, inhalation, or transdermal application.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. For osteoarthritis, IL-RL is preferably administered directly into the affected joint by injection or during arthroscopic examination. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above. For introduction of nucleic acids encoding IL-17RL into prostate cancers, particularly into lesions that have metastasized into bone, direct injection of the nucleic acids or of the vector into the tumor is preferred.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of IL-17B, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

In one embodiment, IL-17B modulators are administered to a patient to correct local bone or cartilage pathologies in the form of a tissue engineering device. Tissue engineering devices comprise a therapeutic molecule and an insoluble carrier matrix, and are implanted at the site of injury or disease. See Reddi, Nature Biotechnology 16(3):247-52 (1998). Insoluble carrier matrices restrict delivery of the chondroleukin modulator to the site of injury or disease, extend the half-life of therapeutic molecules, or provide a substrate for chondrogenic, osteogenic, or angiogenic cells migrating into the site of repair. Cells, proteins, small molecules, and polynucleotides may all be embedded in insoluble carrier matrices for delivery of chondroleukin modulators.

Numerous insoluble carrier matrices suitable for delivery of chondrogenic and osteogenic agents are known in the art. See Kirker-Head, Adv Drug Deliv Rev 43:65-92 (2000). In general, desirable matrices are non-immunogenic, non-toxic, bioabsorbable, malleable, steriliazable, and easily manufactured, although the ideal properties of a matrix depend on the therapeutic agent and site of implantation. Collagen is a suitable carrier matrix, as described in the Examples. Demineralized bone matrix, polylactides and polyglycolides, hydrogels, fibrin, clotted blood, hydroxyapatite, calcium phosphates, titanium, and combinations thereof, are also suitable insoluble carrier matrices for practice of the invention.

Optimal dosage of a chondroleukin modulator may be determined by the concentration required to achieve the desired inhibition or enhancement of chondroleukin activity in the in vivo chondrogenic assays described herein. For administration to a human, chondroleukin modulators are generally administered at a dosage from 1 μg to 10 mg, 10 μg to 1 mg, or 100 μg.

### THERAPEUTIC USES OF IL-17B AGONISTS

The formation of cartilage is generally the first step in the process of bone formation. Accordingly, IL-17B may be administered therapeutically to prevent or limit unwanted formation of cartilage or bone. More generally, a discovery of the present invention is the ability of IL-17B to antagonize the anabolic effects of BMPs. Accordingly, IL-17B or IL-17B agonists may be administered whenever inhibition of BMP activity is desired.

Extraskeletal deposition of calcium and phosphate is involved in a variety of disorders; see Whyte, in Seibel et al. eds., Dynamics of Bone and Cartilage Metabolism, Academic Press (1999). In general, ectopic mineralization may be the result of metastatic calcification, dystrophic calcification, or ectopic ossification. The involvement of BMPs in ectopic mineralization indicates that chondroleukin and chondroleukin agonists may be used to treat disorders of ectopic mineralization. For example, BMP action is implicated in ossification of the posterior longitudinal ligament, ossification of the ligamentum flavum, and fibrodysplasia ossificans progressive (Helm et al., Neurosurgery 46(5):1213-22 (2000)). Hence, chondroleukin or chondroleukin agonists may be administered to treat pathogenic ossification.

BMP-mediated cartilage formation also plays a role in vascular calcification associated with atherosclerotic plaques, and proteins such as Matrix GLA Protein that combat vascular calcification may do so by inhibiting BMPs. See Bostrom, Z Kardiol 89 Suppl 2:69-74 (2000) and references within. Chondroleukin is expressed in heart and kidney, suggesting that chondroleukin plays a role in preventing calcification, hypertrophy, or fibrosis of the heart and kidney. chondroleukin or chondroleukin agonists may therefore be administered to heart or blood vessel walls, either by local or systemic delivery, to prevent or reverse the formation of atherosclerotic plaques or hypertrophy. Likewise, chondroleukin and chondroleukin agonists may be administered systemically to treat fibrosis of the kidney.

Chondroleukin and chondroleukin agonists may also be used to limit the extent of bone growth in therapeutic bone regeneration. Excessive bone formation, spreading outside the original bone contour, complicates the use of BMP-impregnated devices to stimulate bone and cartilage growth. See Groeneveld & Burger, Eur J Endocrinology 142:9-21 (2000). Chondroleukin's ability to block BMP-induced cartilage formation allows more precise sculpting of bone and cartilage in vivo and in vitro. A device impregnated with BMPs creates a focus of bone formation, while a device impregnated with chondroleukin creates a zone in which bone formation is inhibited. Thus, appropriate placement of chondroleukin-impregnated devices may be used to locally restrict the spread of BMP-induced bone formation.

### THERAPEUTIC USES OF CHONDROLEUKIN ANTAGONISTS

Degenerative Cartilage Disorders

IL-17B antagonists such as IL-17RL are useful in the treatment of cartilage degeneration associated with osteoarthritis (OA) and rheumatoid arthritis (RA). Although OA and RA arise by distinct etiologies, cartilage and bone remodeling are part of the pathogenic process for both diseases. Degenerative cartilage disorders are characterized by an imbalance between anabolic and catabolic factors that remodel cartilage. Accordingly, inhibition of chondroleukin's catabolic effects on bone and cartilage is useful in the treatment of OA, RA and other degenerative cartilage disorders. Degenerative cartilage disorders may also be diagnosed by obtaining a sample of tissue—e.g., from the synovial fluid of a joint—and screening for elevated levels of chondroleukin protein or chondroleukin polynucleotides.

As discussed herein, while chondroleukin inhibitors may be administered systemically or locally, local administration of chondroleukin inhibitors to sites of cartilage degeneration is a preferred treatment for degenerative cartilage disorders. Where the joint tissue is essentially intact, administration of chondroleukin inhibitors such as IL-17RL into the synovial space, or by transformed synoviocytes, is an effective means to reverse cartilage erosion and promote cartilage regeneration. Where cartilage damage has been more extensive, chondroleukin inhibitors are most effectively employed in combination with tissue engineering procedures, in which transplanted cells or tissue engineering devices are implanted to reconstruct the articular cartilage. In both cases, arthroscopic procedures are a preferred method of administering chondroleukin inhibitors. Chondroleukin inhibitors may be administered on their own to treat degenerative cartilage disorders, or in conjunction with established therapies such as tissue transplants and anti-inflammatory regimes (see Chikanza & Fernandes, Expert Opin Investig Drugs 9(7):1499-510 (2000)).

It is a discovery of the present invention that IL-17B synergistically potentiates the catabolic effect of IL-1β on cartilage explant cultures (see Van den Berg, Arthritis Res 3: 18-26 (2001)). When administered along with a suboptimal dose of IL-1β (10 pg/ml), increasing suboptimal dosages of chondroleukin induce proteolgycan degradation and release under conditions where no cartilage degradation would be observed with chondroleukin alone or IL-1β alone. The synergistic catabolic effects of chondroleukin and IL-1β indicate that chondroleukin antagonists are effective to treat degenerative cartilage diseases characterized by IL-1β-induced catabolism. Since chondroleukin and IL-1β work synergistically, combinations of chondroleukin inhibitors and IL-1 inhibitors are more effective in treating degnerative cartilage disorders than either class of inhibitors alone. Accordingly, chondroleukin inhibitors may be administered in combination therapy with IL-1 antagonists such as IRAP or soluble IL-1 receptors; see Evans et al., Clin Orthop 3:S214-9 (2000).

BMP Augmentation

Bone morphogenetic proteins have widespread uses in the treatment of cartilage, bone and neurosurgical pathologies. See Ripamonti & Reddi, Crit Rev Oral Biol Med 8(2): 154-63 (1997); Helm et al., Neurosurgery 46(5):1213-22 (2000); Kirker-Head, Adv Drug Deliv Rev 43:65-92 (2000). However, some applications of BMPs have been limited by the presence in tissues of previously uncharacterized By inhibitors. See Groeneveld & Burger, Eur J Endocrinology 142:9-21 (2000). A discovery of the present invention is that chondroleukin and BMPs have opposite effects on cartilage metabolism, chondroleukin being catabolic for cartilage while BMPs act anabolically. Since cartilage formation is the first step in growth or regeneration of bone, chondroleukin's ability to retard BMP-induced cartilage formation indicates that chondroleukin acts as an endogenous inhibitor of BMP-dependent bone induction in vivo. Inhibitors of chondroleukin reverse this effect. Accordingly, chondroleukin inhibitors may be employed to augment BMP effectiveness in all contexts where BMPs are employed. These contexts include spinal arthrodesis, congenital or trauma-induced cranial defects, long bone defects and non-union fractures, craniofacial reconstruction, and periodontal regeneration. A chondroleukin inhibitor may be conveniently co-administered with a BMP in the same form as the BMP itself—e.g., as a purified protein, a nucleic acid, or secreted from a cell transformed ex vivo. Alternatively, where a therapeutic composition containing BMPs or other anabolic factors is prepared from natural sources and contains endogenous IL-17B (e.g., demineralized bone matrix), chondroleukin may be depleted from the composition by immunoabsorption with the anti-IL-17B antibodies described herein.

### EXAMPLES

The following examples are offered to illustrate, but not to limit the claimed invention.

### Example 1

This Example sets forth the materials and methods used in discovering IL-17RL,

GenBank Database Searching: A systematic search of the GenBank EST database was done using the public BLAST search algorithms. A series of ESTs were identified that had limited homology to the interleukin-17 receptor mRNA cytoplasmic domain. These sequences were assembled using AssemblyLign and MacVector (Accelrys Inc., San Diego Calif.) to create a consensus sequence. The consensus sequence was used to search for additional overlapping human EST sequences, and this process was repeated until no further ESTs could be identified. The consensus sequence was compared to the human genomic DNA working draft sequences, including BAC clone AC018809 and others. This alignment was used to identify the intron-exon borders and the differentially spliced isoforms, and MacVector was used to identify and translate the open reading frames. Similar methods were used to identify the murine, bovine, and chicken homologs. MacVector, AssemblyLign, and the programs in the public domain (BLAST, PredictProtein) were used to predict the protein sequences, homology searches, and protein analysis. NetPhos2.0 (www.cbs.dtu.dk) was used to identify and score all possible cytoplasmic serine, threonine and tyrosine phosphorylation sites, and scores greater than 0.90 were considered probable sites of potential phosphorylation.

Northern blots: Total RNA was isolated from homogenized tissues using RNeasy mini-columns (Qiagen, Valencia Calif.), or purchased from Clontech (Palo Alto, Calif.), electrophoresed on 1% agarose gels, transferred to Hybond N+ membrane (Amersham Pharmacia Biotech, Piscataway N.J.), and UV crosslinked. Two micrograms of mRNA was isolated from primary human chondrocytes, and processed as described above. The Multiple Tissue Northern Filter (Clontech), has 1 μg mRNA from various human tissues in each lane. Probes were made using Random-PrimeIt II (Stratagene San Diego Calif.) using full length 2402 bp cDNA. Prehybridization was done at 68° C. for 30 minutes, the hybridization was performed overnight at 65° C. in 0.2M sodium acetate pH 7.2, 7% SDS, 1 mM EDTA, and 0.5% bovine serum albumin. Blots were washed in 2×SSC/0.5% SDS four times at room temperature, then once for 30 minutes in 0.2×SSC/0.1% SDS at 60° C. and exposed overnight to a Molecular Dynamics (Amersham Pharmacia Biotech) phosphor-storage screen, and finally were digitized on a Molecular Dynamics Storm machine at 100 μm resolution.

Quantitative RT-PCR: Synthetic oligonucleotide primers were designed to specifically amplify portions of exons 1, 7, 12, 14, and 18, using PrimerExpress-1.7 software (Applied Biosystems, Foster City Calif.). cDNA was made from 2 μg random primed total RNA using TaqMan Reverse Transcription reagent kits (Applied Biosystems) and with ThermoScript Reverse Transcription System (Invitrogen Life Technologies, Carlsbad Calif.). Real-time quantitative RT-PCR was done on an ABI 7700 Sequence Detector using Sybr-Green and GAPDH TaqMan reagents (Applied Biosystems) following recommended protocols. Reverse transcription reactions were performed several times and analyzed by real-time quantitative PCR in triplicate. Results were normalized to GAPDH levels using the recommended ΔCt method. For comparison of exon usage across RNA from tissues, a plasmid containing the full length cDNA was used as a control since each plasmid has exactly one copy of each exon. Student's t-tests were performed to assess statistical significance.

Antibody Production: Rabbit polyclonal antibodies were made against synthetic peptides QPRYEKELNHTQQLPDC (SEQ ID NO:28) and DSYFHPPGTPAPGRC (SEQ ID NO:29), corresponding to portions of IL17-RL translated from exons 6 and 19. These antibodies will be sometimes referred to herein as the anti-extracellular domain (“ECD”) and anti-cytoplasmic (“CYTO”) domain antibodies, respectively. The peptides were coupled to SulfoLink beads (Pierce/Endogen, Rockford Ill.) and were used to affinity purify antibodies from the rabbit serum as recommended by the manufacturer. Purified antibodies were diluted to 2 μg/ml for western blotting and 30 μg/ml for immunohistochemistry.

Western Blotting: A 50 mg sample from a human prostate was homogenized in reducing SDS-PAGE sample buffer, boiled for 5 minutes, and then electrophoresed on pre-cast 4-12% gradient gels (Invitrogen Life Technologies). After transfer to Immobilon-P (Millipore, Bedford Mass.), the membranes were blocked with 2% BSA then incubated with the anti-ECD or anti-CYTO antibodies at 2 μg/ml. Horeseradish-peroxidase conjugated goat anti-rabbit secondary antibodies and ECL chemiluminescence (Amersham Pharmacia Biotech) were used for detection.

Immunohistochemistry: Arrays of various human tissues (Imgenex, San Diego Calif.) were incubated with 30 μg/ml of either anti-ECD, anti-CYTO, or normal rabbit IgG, followed by FITC-conjugated anti-rabbit. Slides were mounted in VectaShield (Vector Laboratories, Burlingame Calif.) containing the nuclear stain propidium iodide. Digital images were acquired on a Zeiss LSM-510 confocal microscope.

### Example 2

This Example sets forth the results of the studies using the materials and methods set forth above.

Genomic Structure Reveals Alternative Splicing: Database searches revealed 108 human ESTs with overlapping sequences, from which a continuous consensus sequence of 2402 base pairs was assembled. Alignment against the working draft of the human genome indicated that the gene is comprised of 19 exons located on chromosome 3, and spans 16550 base pairs within the chromosomal region 3p25.3 to 3.24.1 (FIG. 1A). Each intron is flanked by consensus splice acceptor and splice donor sequences with the exception of exon 1, which is not preceded by a splice acceptor, and exon 19, which was not followed by a splice donor. Extensive use of alternative splicing was revealed by the alignment of the ESTs with the consensus full length 2402 bp cDNA sequence. Exons 7, 12, 14, 15, and 18 are frequently spliced out. In addition, exons 6, 8, 9, 11, 14, 18 and 19 have alternative splice donor and acceptor sites that are used in several ESTs from different libraries (FIG. 1B). The full length 2402 bp cDNA consists of exons 1-19 as shown in FIG. 2.

Full Length Protein Translation Predicts Type I transmembrane Protein: The first AUG is at position 205 in exon 1, followed by a 2157 bp open reading frame and a 37 bp 3′ untranslated region with a consensus polyadenylation signal. The translation of this open reading frame yields a 720 amino acid protein, which will be referred to from now on as full length to distinguish it from the numerous predicted shorter protein sequences from alternatively spliced mRNAs. Computer analysis of the fall length amino acid sequence predicts that the initiation methionine is followed by a stretch of 20 hydrophobic amino acids with a consensus signal peptide cleavage site at position 21. The full length mature protein consists of a 447 residue amino-terminal extracellular domain, followed by a single 21 amino acid hydrophobic alpha-helical transmembrane domain encoded by exon 17, and a 232 amino acid domain predicted to be cytoplasmic (FIG. 2). The calculated molecular weight of the mature full length protein is 76,378 Daltons. The acidic extracellular domain has a predicted isoelectric point of 4.71, with nine potential sites for N-linked glycosylation. The 22 cysteines, 66 leucines and 38 prolines in the extracellular domain have the potential for forming extensive secondary structure. The 232 amino acid cytoplasmic domain contains 20 arginines, 6 histidines, and 4 lysines which contribute to its basic isoelectric point of 10.04. It also contains 16 serines, 5 threonines and 3 tyrosines. Of these, 4 serines and 2 threonines were predicted by NetPhos software to be in a context where they may be phosphorylated by intracellular kinases (see methods of Example 1). There are no SH2 or SH3 domains, nor is the cytoplasmic domain predicted to have any kinase activity of its own.

Protein Homology to IL-17 Receptor: Overall this protein is 22% identical and 34% similar to human IL-17Rhl (GenBank accession number NM_{—}014339). The cytoplasmic domains of these proteins are more conserved, sharing 25% identity and 41% similarity across their membrane-proximal 233 amino acids.

Sequence Conservation: The murine homolog (SEQ ID NO:75) of the full length protein was identified by assembly of mouse ESTs. It is 699 amino acids in length, and shares 66% identity and 75% similarity to the human protein. Two of the potentially phosphorylated serines in the cytoplasmic domain are conserved in the mouse homolog, as are 7 of the 9 N-glycosylation sites in the extracellular domain. In addition, several bovine, rat, and chicken ESTs were identified that, when translated, showed a similar degree of homology to the human protein.

Splice Variants and Predicted Protein Products: The EST database contains many ESTs that align to the full length sequence but are missing certain exons. Exons 7,12,14, and 15 are frequently spliced out, and there is evidence that these exons can be spliced out in combinations, leading to a great number of possible protein translations. Since no EST covered the entire 2402 base pair full length cDNA, there are more possibilities for combining exon usage than reported here. For example, one EST (accession number AI078128) sequence begins at the Not-I site in exon 17, then continues through exons 16, 13, 11, 10, 9, and most of exon 8 of the full length cDNA, indicating that exons 12, 14 and 15 were spliced out. If it is assumed that the mRNA from which this was derived contains exons 1 through 8, then this mRNA would be translated into a protein of 372 amino acids. Deletion of exons 14 and 15 introduces a frame-shift so that the protein would contain the secretion signal peptide and most of the extracellular domain including all 9 glycosylation sites, but not the transmembrane or cytoplasmic domains. From the existing ESTs in the database, at least 12 different proteins can be made from this gene (FIG. 1C). The translated proteins fall into two major categories, each with minor variations. The first category includes full extracellular domain transmembrane proteins with intact phosphorylation sites in the cytoplasmic tail. The second includes soluble secreted proteins without transmembrane or cytoplasmic domains.

Gene Expression: Northern blot analysis of 5 μg total RNA isolated from various human tissues shows that the mRNA is strongly expressed in prostate, kidney and trachea. Northern blot analysis of 2 μg mRNA from various human tissues shows one major band with a mobility of approximately 2.5 kb, with some diffuse bands between 2.1 and 3.1 kb. The highest expression was in prostate, liver, kidney, muscle and heart. Intermediate expression was in cartilage, brain, colon, intestine, placenta and lung, and barely detectable expression was in thymus and peripheral blood leukocytes.

Exon Usage by quantitative RT-PCR: Primers were designed to specifically amplify regions of exons 7, 12, 14-15, and 18-19. These primers were used in real-time quantitative PCR on cDNA made from various commercially available total RNA, or on a plasmid clone of the full length cDNA which has exactly one copy of each exon per plasmid. Detection of PCR products corresponding to exons 12, 14-15, and 18-19 occurred at almost the same cycle for all tissues, indicating that these exons are transcribed in equal numbers in the tissues examined. However, the PCR products from exon 7 were consistently detected 1.6 to 3 cycles later (p=0.0006), which indicates that only about ⅓ to ⅛ of the transcripts which include exons 12-19 also include exon 7. In addition, there is some evidence that in brain tissue exon 7 is used even less often than in liver (p=0.05) or heart (=0.06) tissues. These experiments were repeated with cDNA made from the same RNA but using a different reverse-transcripts enzyme system at a different temperature in order to reduce the possibility that the measured differences stemmed from inefficient reverse transcription reactions rather than differential use of exons. Controls with a full length plasmid template (containing an equal copy number of each exon) returned equal values for each primer set across many dilutions. These controls shout that the measured differences in exon usage are indeed real and not artifacts of a specific reverse transcription system, or of the TaqMan detection primers' amplification efficiency.

Immunoblotting: A human prostate biopsy was homogenized, electrophoresed on SDS-PAGE, and analyzed by western blotting with affinity purified antibodies directed against the extracellular and cytoplasmic domains of IL17-RL. The anti-ECD antibody, directed against the amino terminus of the protein, detected multiple bands ranging in size from approximately 33 kDa to almost 60 kDa, confirming the predicted presence of multiple isoforms of IL17-RL. The larger of these bands were also detected by anti-CYTO, which is directed against the carboxy-terminal cytoplasmic domain of IL17-RL.

Immunohistochemistry: Immunohistochemal analysis was performed on arrays of human tissue biopsies using both anti-ECD and anti-CYTO antibodies. Strong reactivity was found with anti-ECD, which is directed against the N-terminal extracellular domain of IL17-RL, in skeletal muscle, prostate, kidney, and placenta. Skeletal muscle showed low reactivity with anti-CYTO, which is directed against the C-terminal cytoplasmic domain of IL17-RL. Prostate, kidney, and placenta showed weak, intermediate, and strong reactivity with anti-CYTO, respectively. Immunohistochemistry was performed on arrays of human prostate cancers using anti-CYTO antiodies. Higher grade carcinomas tended to show decreased reactivity in epithelial cells and an increased reactivity in stromal regions relative to normal prostate.

Genbank: The protein sequences described above have been annotated in the GenBank database as protein MGC10763 (human) and MGC6973 (mouse).

### Example 3

This Example provides exemplary assays for measuring binding of IL-17 to IL-17RL or variants thereof.

A. Surface Plasmon Resonance (BIAcore) Assays

The BIAcore is a optical biosensor that utilizes surface plasmon resonance to measure on rates and off rates of a bi-molecular reaction (Jonsson, U et al., Biotechniques, 11:620 (1991), Karlsson, R., et al., J. Immunological Methods 145(1-2):229-40 (1991)). The disassociation constant (Kd), also referred to as the affinity constant is derived by dividing the off rate by the on rate.

Briefly, the extracellular domain (ECD) of IL-17RL is covalently bound to the BIAcore sensor chip. An IL-17, such as IL-17B, is flowed past the immobilized receptor and changes in surface plasmon resonance, measured in response units (RU) is monitored. A series of 6 two-fold serial dilutions are tested. The linear portion of the response is analyzed to determine the on rate. Thereafter the same buffer without the IL-17 is flowed past the sensor chip and the linear portion of the decreasing response rate is analyzed to determine the off rate. The affinity constant is then calculated.

The opposite experiment is also performed where an IL-17 is immobilized on the sensor chip and serial dilutions of IL-17RL are flowed past and on rates and off rates are measured. An advantage of the BIAcore system is that no labeling is needed for detection, allowing the measurement of binding of the unaltered proteins.

B. Flow Cytometry Assays

Binding can also be measured using a flow cytometer. Briefly, human 293 cells are transiently co-transfected with expression vectors for green fluorescent protein (GFP) and full length IL-17RL. After 24 hours the cells are incubated with an IL-17, such as IL-17B. Binding is revealed using phosphotidylethanolamine (PE) conjugated antibodies raised against IL-17B. Binding of the antibodies to the ligands which are bound to the transmembrane receptors in the GFP co-transfected population of cells is measured using a flow cytometer.

### Example 4

### Purification and Cloning of Chondroleukin

This Example sets forth the methods used to discover chondroleukin.

Sample collection, extraction, separation and analysis. Articular cartilage (2 kg) was dissected from the ankle joints of calves. The cartilage was finely chopped and extracted with 1.2 M guanidine hydrochloride. 0.2% CHAPS, 1 mM ethylenediamine-tetraacetic acid (EDTA), 10 mM phenylmethylsulfonylfluoride (PMSF), pH 7.2, overnight at 4° C. The extract was buffer exchanged with 6M urea, freshly deionized using AG-501 X8 (D) resin (BioRad), 0.05M Tris-HCl, 0.15M NaCl pH 7.2 and loaded onto a 1L SP strong cation exchange column (Amersham/Pharmacia) and eluted stepwise with concentrations of 0.25 M, 0.5M and 1.0M NaCl in the same buffer. The 0.5 M NaCl eluate from the SP column was diluted 10:1 with 0.1% aqueous Trifluoroacetic acid (TFA) and fractionated by preparative (22×250 mm) C-4 reverse phase HPLC (Vydac). A gradient of 0 to 20% B in 5 min, 20-50% B in 30 min and 50-100% B in 3 min was used in which buffer A was 0.1% aqueous TFA and buffer B was 0.1% TFA in acetonitrile. The flow rate was 10 ml/min and 5 ml fractions were collected every 30 seconds. Fractions 19 and 20 (eluting from 9 to 10 minutes into the run) were each re-fractionated by analytical (2.1×250 mm) C4 reverse phase HPLC using the same gradient with a flow rate of 1 ml/min and 1 ml fractions were collected.

SDS-PAGE and 2D SDS-PAGE. The HPLC fractions were analyzed by SDS-PAGE using 15% gels (BioRad, Hercules, Calif.) stained with silver stain Novagen). 2D SDS-PAGE was performed using the Multiphor II (Amersham/Pharmacia), an immobilized linear pH gradient of 3-10 for the 1^{st} dimension (3000V for 24 h) and an 8-18% gradient SDS-PAGE for the 2^{nd} dimension (600V for 2 h). Coomassie stain (Bio-Rad) was used for detection.

Tryptic in-gel digest and Edman sequencing. Tryptic in-gel digestion and Edman sequencing of the excised 2D gel spots were performed at the Protein Structure Lab at UC Davis.

In-vivo Chondrogenic activity assay. The HPLC fractions were precipitated onto an inert collagenous carrier composed of 25 mg Insoluble Collagenous Bone Matrix (ICBM), 1 mg Shark Chondroitin Sulfate (Sigma) and 300 mg Rat tail Type I collagen. The pellets were washed three times with ice cold 80% ethanol and dried in a SpeedVac. Pellets for bioassays were placed into subcutaneous pouches made by sterile blunt dissection in the thoracic region of 25-28 day-old Long-Evans rats. After eleven days, the pellets were removed and histological slides were prepared and stained with toluidine blue.

Isolation and purification of chondroleukin. Several cation exchange column-purified, chromatographic fractions of bovine articular cartilage (2 kg) extract demonstrated histological evidence of abundant chondrogenic activity in subcutaneous implants. Aliquots of these fractions were combined, concentrated and analyzed by 2D SDS-PAGE. The sample separated into seven major spots on the Coomassie stained 2D gel. Six spots, when subjected to in-gel tryptic digest followed by Edman amino acid sequencing, yielded tryptic peptides from well-known proteins while the seventh spot yielded 3 novel peptide sequences ranging from 13 to 18 amino acid residues in length (Table 3).

TABLE 3

List of Proteins identified by Edinan sequencing of spots on 2D
gel. Three novel tryptic peptides were found in the analysis of spot
#2. All other spots were identified as well known proteins.

| | | | | |
|---|---|---|---|---|
| Spot # | Description | ˜kDa | ˜pI | Tryptic peptides |

| | | | | |
|---|---|---|---|---|
| 1 | Ribosomal Protein | 10 | 10 | HGRPGIGATHSSR (SEQ ID NO:30) |
| | S15 | | | |
| 2 | Novel Sequences | 21 | 9.5 | AVMETIAVGCTCIF (SEQ ID NO:31), |
| | | | | VPADLPEAQCLCLGCVNP (SEQ ID NO:32), |
| | | | | LSPWGYSINHDPS (SEQ ID NO:33) |
| 3 | C-Type Lectin | 14 | 9.0 | YICEFTIPQ (SEQ ID NO:34) |
| | precursor | | | |
| 4 | Lysozyme C | 10 | 9.5 | TDYGIFQINS (SEQ ID NO:35) |
| 5 | C-Type Lectin | 8 | 9.5 | EMQALQTVCLR (SEQ ID NO:36) |
| | precursor | | | |
| 6 | C-Type Lectin | 14 | 8.5 | SLPGVNDFXLGINEDMVA (SEQ ID NO:37) |
| | precursor | | | |
| 7 | C-Type Lectin | 14 | 8.0 | YICEFTIPQ (SEQ ID NO:38) |
| | precursor | | | |

Yield of chondroleukin in bovine articular cartilage—Two kilograms of bovine articular cartilage was extracted to produce the sample loaded onto the gel and the spot of chondroleukin on the Coomassie stained 2D gel was estimated to be on the order of 50 μg. Applying an estimated 50% loss in the column purifications and buffer exchanges (1L SP cation exchange column, preparative and analytical reverse phase C4 HPLC), gives an estimated yield of 50 μg of chondroleukin per kilogram of bovine articular cartilage.

Degenerate PCR. Forward (5′-GTN CCN GCN GAY YTN CCN GAR GCN CAR TG-3′ (SEQ ID NO:39)) and reverse (5′-TGN TAD CGN CAN CCN ACR TGN ACR TAD AA-3′ (SEQ ID NO:40)) degenerate oligonucleotide primers were designed based on the tryptic peptide sequences VPADLPEAQCLCLGCVNP (SEQ ID NO:32) and AVMETIAVGCTCIF (SEQ ID NO:31) (Table 3) and used to amplify a PCR product from bovine articular chondrocyte cDNA. (Degenerate primers use libraries to account for the fact that multiple codons can encode a single amino acid. The nomenclature used in the primers set forth above is that of the MacVector program and employs the following nomenclature: M=AC, R=AG, N=AT, S=GC, Y=CT, K=GT, V=AGC, H=AGC, H=ACT, D=AGT, B=GCT, N=AGCT.) Bovine articular cartilage was dissected from the metacarpophalangeal joint of a calf (1-3 month). Collagenase (Sigma) digestion of the tissue was followed by total RNA isolation using the RNeasy Mini kit (Qiagen). Oligo (dT) primers were used to prime the mRNA for cDNA synthesis according to the Advantage RT-for-PCR kit protocol (Clontech).

PCR product sequence determination. The ˜200 bp degenerate PCR product was subcloned into the TOPOcloning vector by heat shock and grown O/N under antibiotic selection. 10 colonies were picked and submitted for microsequencing (Davis Sequencing, Davis, Calif.). A consensus sequence was deduced using AssemblyLign software.

EST database searching. The consensus DNA sequence was used to search the NIH BLAST databases of all known proteins (http://www.ncbi.nlm.nih.gov/blast), the human EST databank (http://www.ncbi.nlm.nih.gov/dbEST) was also searched. The DNA sequence of IMAGE clone #783987 (3′) resulted in a 94% match with the bovine PCR product. EST sequences from Soares total fetus, fetal heart, and fetal liver/spleen all matched. The IMAGE number was used to locate the 5′ sequence of clone #783987. The full-length translated EST sequence contained all 3 peptides found in the tryptic digest of the 2D spot from the chondrogenic cartilage fraction. The DNA sequence also contained the in-frame Kozac consensus region, a start codon and a stop codon.

Tissue specificity. Northern blot analysis demonstrated that chondroleukin mRNA is found in cartilage, liver, kidney, heart and skeletal muscle.

### Example 5

### Expression of Recombinant Human Chondroleukin (rhCL)

EST clone #783987 was modified for insertion into an insect constitutive expression vector. PCR was used to add BamH1 and EcoR1 sites and mutate the stop codon to allow expression of the C-terminal Histidine and V5 epitope tags which were contained in the pIZ His/V5c vector (Invitrogen). The putative signal peptide, indicated by the SignalP software was retained in the insect construct, as secretion of the recombinant protein was desired. The PCR product was gel purified and ligated into the vector. The resulting construct was transfected into High Five insect cells using a lipid-mediated transfection reagent, Insectin plus (Invitrogen). Clones were generated using cloning cylinders according to the pIZ protocol (Invitrogen). High Five cells were cultured adherently and then in suspension in Ultimate Insect Serum-Free Medium (Invitrogen). The recombinant protein was recovered from the medium by immobilized metal affinity chromatography (IMAC) or cation exchange chromatography.

### Example 6

### Stimulation of Cartilage Catabolism and Inhibition of BMP Activity by Chondroleukin

Primacy Chondrocyte isolation. Articular cartilage was dissected from the metacarpophalangeal joints of newborn calves. For each experiment, tissue was harvested from a single animal. The cartilage was minced, washed three times in serum-free DMEM:F12 medium containing 1% penicillin/streptomycin and then digested for 12 hours in a 0.2% solution of collagenase P in DMEM:F12 medium containing 3% fetal bovine serum in a 37° C. shaking water bath. The solution containing the isolated cells was filtered twice through 70 μm mesh, washed two times in serum-free DMEM:F12 medium and recovered by centrifugation at 1,000 rpm at 4° C. The cell pellet was resuspended in culture medium and counted using a hematocytometer.

Primary Chondrocyte cultures. The chondrocytes were cultured in monolayer at 13,000 cells per cm² in DMEM:F12 medium supplemented with 50 μg/ml (1-ascorbic acid phosphate, 100 μg/ml sodium pyruvate, 1% (vol/vol) penicillin-streptomycin, and 1% fetal bovine serum).

Articular Chondrocyte Explant culture. 30 to 40 explant disks (2 mm×2-3 mm) were prepared from the articular cartilage of a single calf (1 to 3 months) metacarpophalangeal joint as described (Luyten et al., Journal of Biological Chemistry 267(6):3691-5 (1992)). The disks were washed with DMEM:F12 +1× Penicillin, Streptomycin, and Neomycin (PSN), serum free. 150 mg (3 disks) were maintained in each well of a 12 well plate in 1 ml of basal medium (DMEM:F12, 0.2% Bovine Serum Albumin (BSA), 1×PSN, 50 μg/ml gentamicin, 50 μg/ml Ascorbic Acid and 1 μg/ml Fungizone) for 6 days at 37° C. in a 5% CO₂ incubator with a change of medium every 3 days.

Proteoglycan Synthesis Assay. 12 well plates were seeded with 3 explant disks/well in 1 ml basal medium for the explant assay and 250,000 primary culture chondrocytes/well in 1 ml basal medium containing 1% FBS for the monolayer assay (Lietman et al., J Bone Joint Surg Am 79(8): 1132-7 (1997)).

One hour prior to labeling, the medium was replaced with 0.9 ml of basal medium containing either rhBMP-7, recombinant chondroleukin, or rhBMP-7 and recombinant chondroleukin together. Labeling was accomplished by adding 40 μCi ³⁵SO₄ in 0.1 ml of basal medium to each well and incubating for 4 hours at 37° C. Each well was washed twice with 2 ml medium and then twice with 2 ml of 10 mM EDTA, 10 mM sodium sulfate, 0.1M sodium phosphate, pH 6.5. The explant cultures were next digested for 3 hours at 55 ° C. with proteinase K. The digestion reaction was centrifuged and 0.2 ml of the supernatant was diluted with 2.3 ml of elution buffer (4M guanidine-HCl, 50 mM sodium acetate, 0.5% Triton X-100, pH 6.0). PD-10 columns (Pharmacia) equilibrated with elution buffer, were loaded with 2.5 ml of diluted digestion reaction and eluted in seven 0.5 ml fractions. 10 ml of scintillation cocktail was added to each sample before counting on an LS-6500 scintillation counter (Beckman). PicoGreen (Molecular Probes) was used according to manufacturer's instructions, measured on the Storm phosphoimager (Molecular Dynamics) to determine DNA concentration, and used to normalize the data from the scintillation counter.

Proteoglycan Release Assay. After 6 days of equilibration (described above) the explant cultures were labeled for 24 hours with 40 μCi/ml ³⁵SO₄ in basal medium. Cultures were then washed 3 times with basal medium and maintained for 2 additional days with 1 change of basal medium to remove any unincorporated isotope. The explants were then placed in a 12 well plate with 3 explants/well. The explant culture was fed with serum free basal medium containing either no additive (control), or rhBMP-7 and recombinant chondroleukin, separately and in combination. The cultures were fed on day 0, day 4, and day 8. Conditioned medium was collected on day 4 and 8 before feeding and on day 12 at the termination of the experiment.

### Example 7

### Generation of Anti-Chondroleukin Antibodies

Peptides (CVPADLPEAQCLCLGCVNP (SEQ ID NO:41) and CAVMETIAVGCTCIF (SEQ ID NO:42)) were synthesized based on the tryptic fragments identified in Example 4. A cysteine was added to the N-terminal of each peptide to allow convenient conjugation to keyhole limpet hemocyanin (KLH). Each KLH/peptide conjugate was used to hyperimmunize 2 rabbits. The test bleeds were assayed by immobilized peptide ELISA using the pre-immunization bleed as a negative control. Peptides were immobilized on SulfoLink resin (Pierce) and used to purify anti-peptide IgG from the raw serum. These purified polyclonal antibodies were used to detect native and recombinant chondroleukin by immunoblotting and immunohistochemical procedures. An N-terminal antibody was also generated by first synthesizing a peptide (CQPRSPKSKRKGQGRP (SEQ ID NO:43)) based on a unique chondroleukin specific sequence that does not cross react with IL-17. This antibody can be used as a component in a ELISA specific for chondroleukin in patient fluid samples or tissue culture media samples to determine the concentration of chondroleukin in those samples.

### Example 8

### Cloning and Expression of a Soluble Chondroleukin Receptor

To express the extracellular domain of the chondroleukin receptor, EST ID# 2985728 (5′) (accession #AW675096) in the pCMV-sport6 vector, was obtained from the ATCC. The coding sequence of the chondroleukin receptor was confirmed by amplification of the EST with the following PCR primers:

(SEQ ID NO:44)

| | |
|---|---|
| forward | ATGTCGCTCGTCCTGCTAAGCCTGG |
| and | |

(SEQ ID NO:45)

| | |
|---|---|
| reverse | TTAAAGGCAAGGGGAAGAGTCTTGAGAGTTCTCA. |

A second set of primers was designed to clone the soluble chondroleukin receptor(sCLR) into the Pichia pastoris expression vector pPICZα (Invitrogen). The CLR signal peptide comprised of the N-terminal 17 amino acids was deleted and replaced by the yeast signal peptide included in pPICZα. The forward primer used to delete the signal peptide and reconstruct a EcoR1 site and the yeast signal peptide had the sequence. GCGAATTCCGAGAGCCGACCGTTCAA (SEQ ID NO:46).

Two reverse primers were designed. Both were designed to eliminate the transmembrane and cytosolic portion of the transmembrane receptor. The first primer contained a stop codon at the end of the extracellular coding sequence. The second primer lacked a stop codon, yielding a fusion protein with the c-myc antibody epitope and polyhistidine tag encoded by pPICZα fused to the C-terminus of sCLR. The reverse primer containing a stop codon directly following the extracellular coding sequence was GCTCTAGATTAAGCAGTGATGTTCGGATCCCACA (SEQ ID NO:47), while the reverse primer allowing translation of the pPICZα C-terminal tags was GGTCTAGAGCAGCAGTGATGTTCGGATCCCACA (SEQ ID NO:48). Both of the reverse primers contained an Xba1 restriction site.

The IL-17RL extracellular coding sequence was amplified from the EST 2985728 plasmid, employing the forward primer and a reverse primer either lacking or containing a stop codon. The amplification products and pPICZα were each digested with EcoR1 and Xba1, gel purified and ligated separately to create sCLR expression plasmids either with or without a C-terminal c-myc tag and a poly histidine tag.

The constructs were electroporated into E. coli and propagated. The plasmid was purified from E. coli using a Qiagen plasmid prep kit. The purified plasmid was then linearized using a unique Sac1 site. The linearized plasmid was electroporated into the KM71H and X33 strains of the yeast Pichia pastoris (Invitrogen) and selected for Zeocin resistance on YPD plates containing 1M sorbitol. Colonies were picked after 3 days growth and screened for recombinant sCLR protein expression. Positive colonies were used for scaled-up protein expression. An immobilized nickel column was used to purify the tagged version of sCLR, and anti-c-myc antibodies were used to detect the recombinant protein by immunoblotting. Polyclonal antibodies, directed against the extracellular CLR amino acid sequence CKKNEETVEVNFTTTPLGNR (SEQ ID NO:49), were purified by immobilized peptide affinity chromatography and used to detect native CLR and the untagged recombinant human sCLR.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A nucleic acid encoding a polypeptide with 85% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F.

2. A nucleic acid of claim 1, wherein the nucleic acid encodes a polypeptide with 90% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

3. A nucleic acid of claim 1, wherein the nucleic acid encodes a polypeptide with 95% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

4. A nucleic acid of claim 1, wherein the nucleic acid encodes a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

5. An expression cassette comprising a promoter operatively linked to a nucleic acid of claim 1.

6. An expression cassette comprising a promoter operatively linked to a nucleic acid of claim 2.

7. An expression cassette comprising a promoter operatively linked to a nucleic acid of claim 3.

8. An expression cassette comprising a promoter operatively linked to a nucleic acid of claim 4.

9. A host cell comprising an expression cassette of claim 5.

10. A host cell comprising an expression cassette of claim 6.

11. A host cell comprising an expression cassette of claim 7.

12. A host cell comprising an expression cassette of claim 8.

13. A host cell of claim 9, wherein the host cell is selected from the group consisting of a chondrocyte, a synoviocyte, and a mesenchymal stem cell.

14. A host cell of claim 10, wherein the host cell is selected from the group consisting of a chondrocyte, a synoviocyte, and a mesenchymal stem cell.

15. A host cell of claim 11, wherein the host cell is selected from the group consisting of a chondrocyte, a synoviocyte, and a mesenchymal stem cell.

16. A host cell of claim 12, wherein the host cell is selected from the group consisting of a chondrocyte, a synoviocyte, and a mesenchymal stem cell.

17. A polypeptide with 85% or greater sequence identity to a polypeptide having a sequence selected from the group consisting of: SEQ ID NO:2, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F.

18. A polypeptide of claim 17, which polypeptide has 90% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

19. A polypeptide of claim 17, which polypeptide has 95% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

20. A polypeptide of claim 17 having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

21. A composition comprising a polypeptide of claim 17 and a pharmaceutically acceptable carrier.

22. A composition comprising a polypeptide of claim 18 and a pharmaceutically acceptable carrier.

23. A composition comprising a polypeptide of claim 19 and a pharmaceutically acceptable carrier.

24. A composition comprising a polypeptide of claim 20 and a pharmaceutically acceptable carrier.

25. A use of a nucleic acid sequence of claim 1 for the manufacture of a medicament to modulate cartilage or bone growth in a mammal.

26. A use of a nucleic acid sequence of claim 2 for the manufacture of a medicament to modulate cartilage or bone growth in a mammal.

27. A use of a nucleic acid sequence of claim 3 for the manufacture of a medicament to modulate cartilage or bone growth in a mammal.

28. A use of a nucleic acid sequence of claim 4 for the manufacture of a medicament to modulate cartilage or bone growth in a mammal.

29. A use of a polypeptide of claim 17 for the manufacture of a medicament to modulate cartilage or bone growth in a mammal.

30. A use of a polypeptide of claim 18 for the manufacture of a medicament to modulate cartilage or bone growth in a mammal.

31. A use of a polypeptide of claim 19 for the manufacture of a medicament to modulate cartilage or bone growth in a mammal.

32. A use of a polypeptide of claim 20 for the manufacture of a medicament to modulate cartilage or bone growth in a mammal.

33. A use of a nucleic acid sequence of claim 1 for the manufacture of a medicament to restore androgen-responsiveness to a prostate cancer cell.

34. A use of a nucleic acid sequence of claim 2 for the manufacture of a medicament to restore androgen-responsiveness to a prostate cancer cell.

35. A use of a nucleic acid sequence of claim 3 for the manufacture of a medicament to restore androgen-responsiveness to a prostate cancer cell.

36. A use of a nucleic acid sequence of claim 4 for the manufacture of a medicament to restore androgen-responsiveness to a prostate cancer cell.

37. A method of decreasing catabolic activity in bone or cartilage in a mammal, said method comprising administering to said mammal a polypeptide with 85% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F, in an amount sufficient to lower levels of said IL-17, thereby decreasing catabolic activity in said bone or cartilage.

38. A method of decreasing catabolic activity in bone or cartilage in a mammal, said method comprising administering to said mammal a polypeptide with 90% or greater sequence identity to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F, in an amount sufficient to lower levels of said IL-17, thereby decreasing catabolic activity in said bone or cartilage.

39. A method of decreasing catabolic activity in bone or cartilage in a mammal, said method comprising administering to said mammal a polypeptide with 95% or greater sequence identity to a polypeptide having a sequence selected from the group consisting of: SEQ ID NO:2, SEQ ID NO:5, SEQ ID NO:7, SEQ ID NO:9, SEQ ID) NO:11, SEQ ID NO:13, SEQ ID NO:15, SEQ ID NO:17, SEQ ID NO:19, SEQ ID NO:21, SEQ ID NO:23, and SEQ ID NO:25, which polypeptide binds to an interleukin-17 (IL-17) selected from the group consisting of IL-17A, IL-17B, IL-17C, IL-17E and IL-17F, in an amount sufficient to lower levels of free IL-17 in said mammal, thereby decreasing catabolic activity in said bone or cartilage.

40. A method of decreasing catabolic activity in bone or cartilage in a mammal, said method comprising administering to said mammal a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25, in an amount sufficient to lower levels of free IL-17 cytokines in said mammal, thereby decreasing catabolic activity in said bone or cartilage.

41. A method of determining the aggressiveness of a prostate cancer cell, said method comprising determining the presence or absence in said cell of an epitope of IL-17RL (SEQ ID NO:1), wherein the determination that said epitope is absent in said cell indicates that the cancer is more aggressive than a like cell in which said epitope is present.

42. A method of claim 41, wherein the detection of the presence or absence of epitope is performed using an antibody which specifically binds said epitope.

43. A method of restoring androgen-responsiveness to a prostate cancer cell, said method comprising administering to said prostate cancer cell a nucleic acid encoding a polypeptide which binds IL-17B, and further wherein the polypeptide has 90% or greater sequence identity to a polypeptide having a sequence selected from the group consisting of: SEQ ID NO:2, SEQ ID NO:17, SEQ ID NO:19, SEQ ID NO:21, SEQ ID NO:23 and SEQ ID NO:25, thereby restoring androgen-responsiveness to said prostate cancer cell.

44. A method of restoring androgen-responsiveness to a prostate cancer cell, said method comprising administering to said prostate cancer cell a nucleic acid encoding a polypeptide which binds IL-17B, and further wherein the polypeptide has 95% or greater sequence identity to a polypeptide having a sequence selected from the group consisting of: SEQ ID NO:2, SEQ ID NO:17, SEQ ID NO:19, SEQ ID NO:21, SEQ ID NO:23 and SEQ ID NO:25, thereby restoring androgen-responsiveness to said prostate cancer cell.

45. A method of restoring androgen-responsiveness to a prostate cancer cell, said method comprising administering to said prostate cancer cell a nucleic acid encoding a polypeptide which binds IL-17B, and further wherein the polypeptide has a sequence selected from the group consisting of: SEQ ID NO:2, SEQ ID NO:17, SEQ ID NO:19, SEQ ID NO:21, SEQ ID NO:23 and SEQ ID NO:25, thereby restoring androgen-responsiveness to said prostate cancer cell.

46. An antibody which binds to a polypeptide having a sequence selected from the group consisting of: SEQ ID NO:2, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, and SEQ ID NO:25.

47. An antibody of claim 46, wherein said antibody binds to an extracellular domain of said polypeptide.

48. A method of stimulating growth of bone or cartilage in a patient with a bone or cartilage pathology, the method comprising administering an IL-17B antagonist to the patient, thereby stimulating the growth of bone or cartilage.

49. The method of claim 48, further comprising the step of administering a tissue graft to the patient.

50. The method of claim 48, further comprising the step of administering a bone or cartilage growth factor to the patient.

51. The method of claim 48, wherein the bone or cartilage growth factor is a bone morphogenetic protein.

52. The method of claim 48, wherein the patient has a degenerative cartilage disorder.

53. The method of claim 48, further comprising the steps of:
(a) obtaining a sample of tissue from the patient, and
(b) measuring the amount of IL-17B in the sample.

54. The method of claim 48, wherein the IL-17B antagonist is a monoclonal antibody.

55. The method of claim 48, wherein the IL-17B antagonist is a soluble IL-17B receptor.

56. The method of claim 55, wherein said soluble IL-17B receptor is an IL-17RL lacking a transmembrane domain.

57. A method of claim 56 wherein said IL-17RL lacking a transmembrane domain is selected from the group consisting of SEQ ID NO:5, SEQ ID NO:7, SEQ ID NO:9, SEQ ID NO:11, SEQ ID NO:13, and SEQ ID NO:15.

58. The method of claim 48, wherein the IL-17B antagonist is a polynucleotide encoding a soluble IL-17B receptor.

59. The method of claim 58, wherein the polynucleotide encodes a soluble IL-17B receptor selected from the group consisting of SEQ ID NO:5, SEQ ID NO:7, SEQ ID NO:9, SEQ ID NO:11, SEQ ID NO:13, and SEQ ID NO:15.

60. The method of claim 48, wherein the IL-17B antagonist is a IL-17B antisense polynucleotide.

61. A method of potentiating the activity of a bone morphogenetic protein in a mammal, the method comprising administering the bone morphogenetic protein and a IL-17B antagonist to the mammal.

62. A method of enhancing the regenerative potential of a tissue graft, comprising transforming cells of the tissue graft with an IL-17B antagonist.

63. A method of inhibiting ossification or calcification in a mammal suffering from pathological ossification or calcification, the method comprising administering IL-17B to the mammal in an amount sufficient to inhibit ossification or calcification.

64. A method of diagnosing a cartilage degenerative disorder in a mammal, comprising the steps of:
(a) obtaining a sample of tissue from the mammal
(b) measuring the amount of IL-17B in the sample, and
(c) comparing the amount of IL-17B in the sample with the amount of IL-17B in a sample of tissue from a mammal known to have a cartilage degenerative disorder.

65. A method of stimulating proteoglycan synthesis by a chondrocyte in culture, the method comprising contacting the chondrocyte with an IL-17B antagonist, such that the rate of proteoglycan synthesis is increased.

66. A method of claim 65, wherein said IL-17B antagonist is a soluble IL-17RL.

67. A method of claim 66, wherein said soluble IL-17RL is selected from the group consisting of SEQ ID NO:5, SEQ ID NO:7, SEQ ID NO:9, SEQ ID NO:11, SEQ ID NO:13, and SEQ ID NO:15

68. A method of inhibiting the rate of proteoglycan synthesis by a chondrocyte in culture, comprising the step of contacting the chondrocyte with IL-17B, such that the rate of proteoglycan synthesis is decreased.

69. A mammalian cell comprising a polynucleotide encoding an IL-17B antagonist, wherein the mammalian cell is selected from the group consisting of a chondrocyte, a synoviocyte, and a mesenchymal stem cell.

70. A composition comprising a cartilage growth factor and an IL-17B antagonist in a pharmaceutically acceptable carrier.

71. A composition comprising an IL-17B antagonist and an insoluble carrier matrix.

72. A use of an IL-17B antagonist selected from the group consisting of an antibody and IL-17RL for the manufacture of a medicament to stimulating growth of bone or cartilage in a patient with a bone or cartilage pathology.

73. A use of claim 72 wherein said IL-17B antagonist is a soluble IL-17RL.

74. A use of claim 73, wherein said soluble IL-17RL is a polypeptide with at least 85% sequence identity to a sequence selected from the group consisting of SEQ ID NO:5, SEQ ID NO:7, SEQ ID NO:9, SEQ ID NO:11, SEQ ID NO:13, and SEQ ID NO:15.

75. A use of an IL-17RL for the manufacture of a medicament to potentiate the activity of a bone morphogenetic protein in a mammal.

76. A use of claim 75, wherein said IL-17RL is a soluble IL-17RL.

77. A use of claim 76, wherein said soluble IL-17RL is selected from the group consisting of SEQ ID NO:5, SEQ ID NO:7, SEQ ID NO:9, SEQ ID NO:11, SEQ ID NO:13, and SEQ ID NO:15.

78. A polypeptide encoded by at least one exon selected from the group consisting of exons 1 to 19 (SEQ ID NOs:55-73) of I17RL (SEQ ID NOs:1), which polypeptide binds to an interleukin-17 (IL-17) selected from the group consisting of IL-17A, IL-17B, IL-17C, IL-17E and IL-17F.

79. A nucleic acid encoding a polypeptide of claim 78.

80. A composition comprising a polypeptide of claim 78 in a pharmaceutically acceptable carrier.

81. A use of a polypeptide of claim 78 for the manufacture of a medicament to decrease catabolic activity in bone or cartilage.

82. A polypeptide with at least 85% identity to SEQ ID NO:75, which polypeptide binds to an interleukin-17 (IL-17) selected from the group consisting of IL-17A, IL-17B, IL-17C, IL-17E and IL-17F.

83. A nucleic acid encoding a polypeptide of claim 82.

84. A nucleic acid of claim 83, which nucleic acid has the coding sequence of SEQ ID NO:74.

85. A use of a polypeptide of claim 82 for the manufacture of a medicament.

86. A use of a nucleic acid of claim 83 for the manufacture of a medicament.


SEQUENCE LISTING (76 sequences)

SEQ ID NO: 1 - DNA, 2163 bp, Homo sapiens
  full length human interleukin 17 (IL-17) receptor-like protein (IL-17RL) cDNA open reading frame translated region
  CDS (1)..(2163): longest IL-17RL protein expressed by exons 1-19
  sig_peptide (1)..(63)
atgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgacttaggggtggaggagcctaggaatgcctctctccaggcccaagtcgtgctctccttccaggcctaccctactgcccgctgcgtcctgctggaggtgcaagtgccctgctgccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgagggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaa

SEQ ID NO: 2 - protein, 720 aa, Homo sapiens
  full length human interleukin 17 (IL-17) receptor-like protein (IL-17RL) translated region
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADLGVEEPRNASLQAQVVLSFQAYPTARCVLLEVQVPCCLVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLRADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 3 - DNA, 2344 bp, Homo sapiens
  sequence encoding full length IL-17RL including untranslated 5′ and 3′ ends
agcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgacttaggggtggaggagcctaggaatgcctctctccaggcccaagtcgtgctctccttccaggcctaccctactgcccgctgcgtcctgctggaggtgcaagtgccctgctgccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgagggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctaccaaaaaaaaaa

SEQ ID NO: 4 - DNA, 1842 bp, Homo sapiens
  cDNA encoding 186 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcccaccgctttcctgggagaacgtcactgtggacgtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 5 - protein, 186 aa, Homo sapiens
  186 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSHRFPGRTSLWT

SEQ ID NO: 6 - DNA, 1853 bp, Homo sapiens
  cDNA encoding 269 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 7 - protein, 269 aa, Homo sapiens
  269 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWLYFTTQQSLHEGSSPWRVLTTRPAVRPVSAAMGR

SEQ ID NO: 8 - DNA, 1227 bp, Homo sapiens
  cDNA encoding 332 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 9 - protein, 332 aa, Homo sapiens
  332 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCQTSWGPCSSLAPRVPGGSKRERSKCPGPFSQPWIATSIPRGLPRRDAGWDQARDLGRGTGLK

SEQ ID NO: 10 - DNA, 2285 bp, Homo sapiens
  cDNA encoding 348 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 11 - protein, 348 aa, Homo sapiens
  348 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGEQLGEAAAAGVLVG

SEQ ID NO: 12 - DNA, 2162 bp, Homo sapiens
  cDNA encoding 372 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacgtgaacagctcggagaagctgcagctgcaggagtgcttgtgggctgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 13 - protein, 372 aa, Homo sapiens
  372 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDVNSSEKLQLQECLWAAMGR

SEQ ID NO: 14 - DNA, 2273 bp, Homo sapiens
  cDNA encoding 409 aa soluble form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 15 - protein, 409 aa, Homo sapiens
  409 aa soluble form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWAEGSSPWRVLTTRPAVRPVSAAMGR

SEQ ID NO: 16 - DNA, 2497 bp, Homo sapiens
  cDNA encoding 553 aa membrane bound form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcggggggtgagtgggagcaagcgctgggcggagggccgcccccggggagccaggcctgtgccagctcacctcttccctccccatctgttttctccggcagcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctacccatgtggccc

SEQ ID NO: 17 - protein, 553 aa, Homo sapiens
  553 aa membrane bound form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGGEWEQALGGGPPPGSQACASSPLPSPSVFSGSGRQGPRGSAPLLSR

SEQ ID NO: 18 - DNA, 2284 bp, Homo sapiens
  cDNA encoding 683 aa membrane bound form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacgtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 19 - protein, 683 aa, Homo sapiens
  683 aa membrane bound form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDVNSSEKLQLQECLWADSLGPLKDDVLLLETRGPQDNRSLCALEPSGCTSLPSKASTLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 20 - DNA, 2314 bp, Homo sapiens
  cDNA encoding 693 aa membrane bound form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 21 - protein, 693 aa, Homo sapiens
  693 aa membrane bound form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 22 - DNA, 2343 bp, Homo sapiens
  cDNA encoding 703 aa membrane bound form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacgtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctcgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 23 - protein, 703 aa, Homo sapiens
  703 aa membrane bound form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 24 - DNA, 2350 bp, Homo sapiens
  cDNA encoding 705 aa membrane bound form of human IL-17RL
ccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcaactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggcgcgggacctggggcgggggacgggacttaaataaaggcagacgctgtttttctaccca

SEQ ID NO: 25 - protein, 705 aa, Homo sapiens
  705 aa membrane bound form of human IL-17RL
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 26 - skipped

SEQ ID NO: 27 - DNA, 2402 bp, Homo sapiens
  full length cDNA of human IL-17RL including additional untranslated 5′ and 3′ regions
ccgtgtggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgacttaggggtggaggagcctaggaatgcctctctccaggcccaagtcgtgctctccttccaggcctaccctactgcccgctgcgtcctgctggaggtgcaagtgccctgctgccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctaccaaaaaaaaaa

SEQ ID NO: 28 - protein, 17 aa, Artificial Sequence
  Description of Artificial Sequence: extracellular (ECD) domain portion of IL-17RL
QPRYEKELNHTQQLPDC

SEQ ID NO: 29 - protein, 15 aa, Artificial Sequence
  Description of Artificial Sequence:cytoplasmic (CYTO) domain portion of IL-17RL
DSYFHPPGTPAPGRC

SEQ ID NO: 30 - protein, 13 aa, Artificial Sequence
  Description of Artificial Sequence:ribosomal protein S15 tryptic peptide
HGRPGIGATHSSR

SEQ ID NO: 31 - protein, 14 aa, Artificial Sequence
  Description of Artificial Sequence:novel sequence tryptic peptide
AVMETIAVGCTCIF

SEQ ID NO: 32 - protein, 18 aa, Artificial Sequence
  Description of Artificial Sequence:novel sequence tryptic peptide
VPADLPEAQCLCLGCVNP

SEQ ID NO: 33 - protein, 13 aa, Artificial Sequence
  Description of Artificial Sequence: novel sequence tryptic peptide
LSPWGYSINHDPS

SEQ ID NO: 34 - protein, 9 aa, Artificial Sequence
  Description of Artificial Sequence:C-type lectin precursor tryptic peptide
YICEFTIPQ

SEQ ID NO: 35 - protein, 10 aa, Artificial Sequence
  Description of Artificial Sequence:lysozyme C tryptic peptide
TDYGIFQINS

SEQ ID NO: 36 - protein, 11 aa, Artificial Sequence
  Description of Artificial Sequence:C-type lectin precursor tryptic peptide
EMQALQTVCLR

SEQ ID NO: 37 - protein, 17 aa, Artificial Sequence
  Description of Artificial Sequence:C-type lectin precursor tryptic peptide
  MOD_RES (9): Xaa = any amino acid
SLPGVNDFXLGINDMVA

SEQ ID NO: 38 - skipped

SEQ ID NO: 39 - DNA, 29 bp, Artificial Sequence
  Description of Artificial Sequence:forward degenerate oligonucleotide primer
  modified_base (1)..(29): n = a, g, c or t
gtnccngcngayytnccngargcncartg

SEQ ID NO: 40 - DNA, 29 bp, Artificial Sequence
  Description of Artificial Sequence:reverse degenerate oligonucleotide primer
  modified_base (1)..(29): n = a, g, c or t
tgntadcgncanccnacrtgnacrtadaa

SEQ ID NO: 41 - protein, 19 aa, Artificial Sequence
  Description of Artificial Sequence: novel sequence tryptic peptide with Cys added to N-terminal
CVPADLPEAQCLCLGCVNP

SEQ ID NO: 42 - protein, 15 aa, Artificial Sequence
  Description of Artificial Sequence: novel sequence tryptic peptide with Cys added to N-terminal
CAVMETIAVGCTCIF

SEQ ID NO: 43 - protein, 16 aa, Artificial Sequence
  Description of Artificial Sequence:unique chondroleukin specific sequence
CQPRSPKSKRKGQGRP

SEQ ID NO: 44 - DNA, 25 bp, Artificial Sequence
  Description of Artificial Sequence:EST amplification forward PCR primer
atgtcgctcgtcctgctaagcctgg

SEQ ID NO: 45 - DNA, 34 bp, Artificial Sequence
  Description of Artificial Sequence:EST amplification reverse PCR primer
ttaaaggcaaggggaagagtcttgagagttctca

SEQ ID NO: 46 - DNA, 26 bp, Artificial Sequence
  Description of Artificial Sequence: forward primer to delete signal peptide and reconstruct EcoR1 site and yeast signal peptide
gcgaattccgagagccgaccgttcaa

SEQ ID NO: 47 - DNA, 34 bp, Artificial Sequence
  Description of Artificial Sequence: reverse primer containing stop codon directly following extracellular coding sequence
gctctagattaagcagtgatgttcggatcccaca

SEQ ID NO: 48 - DNA, 33 bp, Artificial Sequence
  Description of Artificial Sequence: reverse primer allowing translation of pPICZalpha C-terminal tags
ggtctagagcagcagtgatgttcggatcccaca

SEQ ID NO: 49 - protein, 20 aa, Artificial Sequence
  Description of Artificial Sequence: extracellular chondroleukin receptor (CLR) amino acid sequence
CKKNEETVEVNFTTTPLGNR

SEQ ID NO: 50 - protein, 160 aa, Homo sapiens
  mature IL-17B (chondroleukin) after signal peptide cleavage
QPRSPKSKRKGQGRPGPLVPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 51 - protein, 180 aa, Homo sapiens [sequence omitted]

SEQ ID NO: 52 - DNA, 771 bp, Homo sapiens
  IL-17B (chondroleukin) cDNA
aattggccctcgaggccaagaattcggtacgaggctggggttccaggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggtccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagcaaaaaaaaaaaaaaaaaaaaaaaaaatatgcggccgcaagttt

SEQ ID NO: 53 - DNA, 543 bp, Homo sapiens
  IL-17B (chondroleukin) full-length protein cDNA
atggactggcctcacaacctgctgtttcttcttaccatttccatcttcctggggctgggccagcccaggagccccaaaagcaagaggaaggggcaagggcggcctgggcccctggtccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctga

SEQ ID NO: 54 - DNA, 483 bp, Homo sapiens
  IL-17B (chondroleukin) mature protein cDNA
cagcccaggagccccaaaagcaagaggaaggggcaagggcggcctgggcccctggtccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctga

SEQ ID NO: 55 - DNA, 106 bp, Artificial Sequence
  Description of Artificial Sequence:human IL-17RL exon 1
atgcctgtgccctggttcttgctgtccttggcactgggccgaagcccagtggtcctttctctggagaggcttgtggggcctcaggacgctacccactgctctccgg

SEQ ID NO: 56 - DNA, 21 bp, Artificial Sequence
  Description of Artificial Sequence:human IL-17RL exon 2
gcctctcctgccgcctctggg

SEQ ID NO: 57 - DNA, 153 bp, Artificial Sequence
  Description of Artificial Sequence:human IL-17RL exon 3
acagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatg

SEQ ID NO: 58 - DNA, 75 bp, Artificial Sequence
  Description of Artificial Sequence: human IL-17RL exon 4
ggcactgggaagagcctgaagatgaggaaaagtttggaggagcagctgacttaggggtggaggagcctaggaatg

SEQ ID NO: 59 - DNA, 110 bp, Artificial Sequence
  Description of Artificial Sequence: human IL-17RL exon 5
cctctctccaggcccaagtcgtgctctccttccaggcctaccctactgcccgctgcgtcctgctggaggtgcaagtgccctgctgccttgtgcagtttggtcagtctgtg

SEQ ID NO: 60 - DNA, 112 bp, Artificial Sequence
  Description of Artificial Sequence: human IL-17RL exon 6
ggctctgtggtatatgactgcttcgaggctgccctagggagtgaggtacgaatctggtcctatactcagcccaggtacgagaaggaactcaaccacacacagcagctgcctg

SEQ ID NO: 61 - DNA, 45 bp, Artificial Sequence
  Description of Artificial Sequence: human IL-17RL exon 7
actgcagggggctcgaagtctggaacagcatcccgagctgctggg

SEQ ID NO: 62 - DNA, 140 bp, Artificial Sequence
  Description of Artificial Sequence: human IL-17RL exon 8
ccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctg

SEQ ID NO: 63 - DNA, 60 bp, Artificial Sequence
  Description of Artificial Sequence: human IL-17RL exon 9
actggaccgcagatcattaccttgaaccacacagacctggttccctgcctctgtattcag

SEQ ID NO: 64 - DNA, 52 bp, Artificial Sequence
  Description of Artificial Sequence: human IL-17RL exon 10
gtgtggcctctggaacctgactccgttaggacgaacatctgccccttcaggg

SEQ ID NO: 65 - DNA, 185 bp, Artificial Sequence
  Description of Artificial Sequence: human IL-17RL exon 11
aggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggac

SEQ ID NO: 66 - DNA, 51 bp, Artificial Sequence
  Description of Artificial Sequence: human IL-17RL exon 12
aaggttctcgagttcccattgctgaaaggccaccctaacctctgtgttcag

SEQ ID NO: 67 - DNA, 46 bp, Artificial Sequence
  Description of Artificial Sequence: human IL-17RL exon 13
gtgaacagctcggagaagctgcagctgcaggagtgcttgagggctg

SEQ ID NO: 68 - DNA, 122 bp, Artificial Sequence
  Description of Artificial Sequence: human IL-17RL exon 14
actccctggggcctctcaaagacgatgtgctactgttggagacacgaggcccccaggacaacagatccctctgtgccttggaacccagtggctgtacttcactacccagcaaagcctccacg

SEQ ID NO: 69 - DNA, 60 bp, Artificial Sequence
  Description of Artificial Sequence: human IL-17RL exon 15
agggcagctcgccttggagagtacttactacaagacctgcagtcaggccagtgtctgcag

SEQ ID NO: 70 - DNA, 49 bp, Artificial Sequence
  Description of Artificial Sequence: human IL-17RL exon 16
ctatgggacgatgacttgggagcgctatgggcctgccccatggacaaat

SEQ ID NO: 71 - DNA, 96 bp, Artificial Sequence
  Description of Artificial Sequence: human IL-17RL exon 17
acatccacaagcgctgggccctcgtgtggctggcctgcctactctttgccgctgcgctttccctcatcctccttctcaaaaaggatcacgcgaaag

SEQ ID NO: 72 - DNA, 42 bp, Artificial Sequence
  Description of Artificial Sequence: human IL-17RL exon 18
ggtggctgaggctcttgaaacaggacgtccgctcgggggcgg

SEQ ID NO: 73 - DNA, 638 bp, Artificial Sequence
  Description of Artificial Sequence: human IL-17RL exon 19
ccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaa

SEQ ID NO: 74 - DNA, 2331 bp, Mus sp.
  mouse IL-17RL cDNA with untranslated regions
ccaaatcgaaagcackssagctgawactgggcctggagtccaggctcactggagtggggaagcatggctggagaggaattctagcccttgctctctcccagggacacggggctgattgtcagcaggggcgaggggtctgcccccccttgggggggcaggacggggcctcaggcctgggtgctgtccggcacctggaagatgcctgtgtcctggttcctgctgtccttggcactgggccgaaaccctgtggtcgtctctctggagagactgatggagcctcaggacactgcacgctgctctctaggcctctcctgccacctctgggatggtgacgtgctctgcctgcctggaagcctccagtctgccccaggccctgtgctagtgcctacccgcctgcagacggagctggtgctgaggtgtccacagaagacagattgcgccctctgtgtccgtgtggtggtccacttggccgtgcatgggcactgggcagagcctgaagaagctggaaagtctgattcagaactccaggagtctaggaacgcctctctccaggcccaggtggtgctctccttccaggcctaccccatcgcccgctgtgccctgctggaggtccaggtgcccgctgacctggtgcagcctggtcagtccgtgggttctgcggtatttgactgtttcgaggctagtcttggggctgaggtacagatctggtcctacacgaagcccaggtaccagaaagagctcaacctcacacagcagctgcctgactgcaggggtcttgaagtccgggacagcatccagagctgctgggtcctgccctggctcaatgtgtctacagatggtgacaatgtccttctgacactggatgtctctgaggagcaggactttagcttcttactgtacctgcgtccagtcccggatgctctcaaatccttgtggtacaaaaacctgactggacctcagaacattactttaaaccacacagacctggttccctgcctctgcattcaggtgtggtcgctagagccagactctgagagggtcgaattctgccccttccgggaagatcccggtgcacacaggaacctctggcacatagccaggctgcgggtactgtccccaggggtatggcagctagatgcgccttgctgtctgccgggcaaggtaacactgtgctggcaggcaccagaccagagtccctgccagccacttgtgccaccagtgccccagaagaacgccactgtgaatgagccacaagatttccagttggtggcaggccaccccaacctctgtgtccaggtgagcacctgggagaaggttcagctgcaagcgtgcttgtgggctgactccttgggccccttcaaggatgatatgctgttagtggagatgaaaaccggcctcaacaacacatcagtctgtgccttggaacccagtggctgtacaccactgcccagcatggcctccacgagagctgctcgcctgggagaggagttgctgcaagacttccgatcacaccagtgtatgcagctgtggaacgatgacaacatgggatcgctatgggcctgccccatggacaagtacatccacaggcgctgggtcctagtatggctggcctgcctactcttggctgcggcgcttttcttcttcctccttctaaaaaaggaccgcaggaaagcggcccgtggctcccgcacggccttgctcctccactccgccgacggagcgggctacgagcgtctggtgggagcactggcgtccgcgttgagccagatgccactgcgcgtggccgtggacctgtggagccgccgcgagctgagcgcgcacggagccctagcctggttccaccaccagcgacgccgtatcctgcaggagggtggcgtggtaatccttctcttctcgcccgcggccgtggcgcagtgtcagcagtggctgcagctccagacagtggagcccgggccgcatgacgccctcgccgcctggctcagctgcgtgctacccgatttcctgcaaggccgggcgaccggccgctacgtcggggtctacttcgacgggctgctgcacccagactctgtgccctcccssttccgcgtcgccccgctcttctccctgccctcgcagctgccggctttcctggatgcactgcagggaggctgctccacttccscggggcgacccgcggaccgggtggaacgagtgacccagscsctgcggtcccccctggacagctgtacttctagctcggaasccccaggctgctgcgaggaatgggacctgggaccctgcactacactagaataaaagccgatacagtattcctaaaaaaaaaaaaaaaaa

SEQ ID NO: 75 - protein, 698 aa, Mus sp.
  mouse IL-17RL
  MOD_RES (1)..(698): Xaa = any amino acid
MPVSWFLLSLALGRNPVVVSLERLMEPQDTARCSLGLSCHLWDGDVLCLPGSLQSAPGPVLVPTRLQTELVLRCPQKTDCALCVRVVVHLAVHGHWAEPEEAGKSDSELQESRNASLQAQVVLSFQAYPIARCALLEVQVPADLVQPGQSVGSAVFDCFEASLGAEVQIWSYTKPRYQKELNLTQQLPDCRGLEVRDSIQSCWVLPWLNVSTDGDNVLLTLDVSEEQDFSFLLYLRPVPDALKSLWYKNLTGPQNITLNHTDLVPCLCIQVWSLEPDSERVEFCPFREDPGAHRNLWHIARLRVLSPGVWQLDAPCCLPGKVTLCWQAPDQSPCQPLVPPVPQKNATVNEPQDFQLVAGHPNLCVQVSTWEKVQLQACLWADSLGPFKDDMLLVEMKTGLNNTSVCALEPSGCTPLPSMASTRAARLGEELLQDFRSHQCMQLWNDDNMGSLWACPMDKYIHRRWVLVWLACLLLAAALFFFLLLKKDRRKAARGSRTALLLHSADGAGYERLVGALASALSQMPLRVAVDLWSRRELSAHGALAWFHHQRRRILQEGGVVILLFSPAAVAQCQQWLQLQTVEPGPHDALAAWLSCVLPDFLQGRATGRYVGVYFDGLLHPDSVPSXFRVAPLFSLPSQLPAFLDALQGGCSTSXGRPADRVERVTQXLRSPLDSCTSSSEXPGCCEEWDLGPCTTLE

SEQ ID NO: 76 - protein, 720 aa, Homo sapiens
  human IL-17RL translation of coding region of SEQ ID NO:27
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADLGVEEPRNASLQAQVVLSFQAYPTARCVLLEVQVPCCLVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT